(19) United States
(12) Patent Application Publication
(10) Pub. No.: US 2019/0385476 A1
(43) Pub. Date: Dec. 19, 2019

(54) Tacit Motion System and Methods

(71) Applicant: Mohammad Mahdi Sadeghi, Natick, MA (US)

(72) Inventor: Mohammad Mahdi Sadeghi, Natick, MA (US)

(21) Appl. No.: 16/440,977

(22) Filed: Jun. 13, 2019

Related U.S. Application Data
(60) Provisional application No. 62/684,427, filed on Jun. 13, 2018.

(51) Int. Cl. G09B 19/00; A63B 24/00; A61B 5/11

(52) U.S. Cl. CPC G09B 19/003; A63B 24/0062; A61B 5/1123; A63B 24/0075; A63B 2230/65; A63B 2220/836; A63B 2230/08; A63B 2230/50; A63B 2024/0009

(57) ABSTRACT

In an illustrative embodiment, a wearable system for encoding and a method for comparing a tacit motion is provided including at least one base pod, each base pod having a processor and at least one first sensor in communication with the processor, the at least first sensor configured to sense a first motion of a wearer; one or more distributed modules, each distributed module including at least one second sensor for sensing a second motion of the wearer, each second sensor configured to be in communication with the processor of the base pod and to be positioned at a distinct location relative to a body and/or joint; and where each sensor is positioned at a different distinct location relative to a body and/or joint, where coordinated data from each sensor is configured to be used for detecting a tacit motion.

### RELATED APPLICATIONS

This application claims priority to U.S. Provisional Patent Application Ser. No. 62/684,427, entitled “Tacit Motion System and Methods,” filed Jun. 13, 2018. All above identified applications are hereby incorporated by reference in their entireties.

### BACKGROUND

Individual and group activities such as fitness, choreography, gymnastics, include gross and tacit motions that are mimicked by each individual over time and between individuals. There is an abundance of group fitness classes which offer lower costs to trainees as compared to a personal trainer. A common trainer is constantly dividing their attention throughout the group to monitor for proper form and boost motivation of individuals. As group fitness classes grow in class sizes attention of the common trainer is diluted. This leads to trainees being unaware if proper form is being maintained. Further, millions of preventative injuries occur every year by overexertion and poor conditioning and unsupervised physical therapy.

Realtime personal performance data can be an informative and motivating factor for improving an individual's health and performance. Currently a gym franchise, Orange Theory Fitness™ (Boca Raton, Fla.) utilizes heart rate monitors during a fitness session to provide individual feedback on an individual's heart rate during the workout. The heart rate is displayed to the individual so they can adjust their intensity of their exertion to match a particular heart rate exertion zone. A system for tracking and comparing movements can enhance the ability of individuals and trainers to monitor, communicate, compare, and instruct tacit motions in a digital form of body language.

### SUMMARY OF ILLUSTRATIVE EMBODIMENTS

The forgoing general description of the illustrative implementations and the following detailed description thereof are merely exemplary aspects of the teachings of this disclosure, and are not restrictive.

In an exemplary embodiment, a wearable system for encoding and a method for comparing a tacit motion is provided including a base pod having memory and a communication port; one or more distributed modules, each distributed module comprising at least one sensor for sensing a tacit motion of a wearer; and where each distributed module is in communication with the communication port.

A system and method for encoding, tracking, and comparing movements of individuals is provided that can enhance ability of individuals and trainers to monitor, communicate, compare, and instruct tacit motions in a digital form of body language. As used herein, a time series of sensor data and spatial-temporal data defining a tacit motion by a wearer can be considered as an Impression. As used herein, an Xpression can be determined as a derivative of an Impression and can be used to compare Impressions to one or more Xpression standards or signatures. Examples of Xpressions can include using the time series of sensor data and spatial-temporal data to define one or more metrics describing one or more patterns of coordinated sensor data. In an example, an Xpression can include one or more metrics describing one or more patterns of coordinated sensor data between at least one base pod and at least one distributed module. In an example, an attribute of each base pod and distributed module can be used to compare an Xpression to an Impression. In an example, an Xpression can store the one or more metrics in a header including at least one sensor attribute to compare an Xpression to an Impression. Examples of the metrics include but are not limited to spatial differences, angle differences, and temporal differences. In an example, an Xpression standard or signature can define a targeted goal for a wearer of the system to attempt to recreate.

In an exemplary embodiment, a method for communicating a tacit motion includes generating a recording file, generating a Xpression from the recording file, and identifying one or more Xpressions within a recording file. In an exemplary embodiment, generating the Xpression signature includes receiving the recording file including a header, having an attribute of each base pod and distributed module, and the array of aggregate sensor data; identifying, for each aggregate sensor data contemporaneously within the array of aggregate sensor data, one or more patterns of coordinated sensor data across the base pod and the one or more distributed modules; and generating an Xpression signature describing the one or more patterns of coordinated sensor data across the base pod and the one or more distributed modules.

In an exemplary embodiment, identifying one or more Xpressions within a recording file includes receiving a sample recording file including a header, having an attribute of each sample base pod and sample distributed module, and the array of aggregate sensor data, and a library of Xpression signatures, where each Xpression signature includes include one or more metrics describing one or more patterns of coordinated sensor data; performing, based on the attribute of each sample distributed module, dynamic time warping to at least one array(t) of the array of aggregate sensor data of the sample recording file; identifying a pattern of coordinated sensor data for each aggregate sensor data within the array of aggregate sensor data of the sample recording file; comparing the one or more patterns of coordinated sensor data across the base pod and the one or more distributed modules of each Xpression signature of the library of Xpression signatures to the pattern of coordinated sensor data for each aggregate sensor data within the array of aggregate sensor data of the sample recording file; and generating an indicator for each matching Xpression within the sample recording file. In an example, the one or more patterns of coordinated sensor data can be across at least one base pod and at least one distributed module.

In an exemplary embodiment, a wearable system for is provided recording a tacit motion including at least one base pod, each base pod having a processor and at least one first sensor in communication with the processor, the at least first sensor configured to sense a first motion of a wearer; one or more distributed modules, each distributed module comprising at least one second sensor for sensing a second motion of the wearer, each second sensor configured to be in communication with the processor of the base pod and to be positioned at a distinct location relative to a different body part of the wearer; and where each sensor is positioned at a different distinct location relative to the wearer; where coordinated data from each sensor is configured to be used for detecting a tacit motion defined at least in part by the first and second motions of the wearer.

In some implementations, each base pod is configured to receive a recording file including an attribute of each base pod and each distributed module, and an array of aggregate sensor data associated with the tacit motion, one or more metrics associated with an Xpression; identify, for each aggregate sensor data contemporaneously within the array of aggregate sensor data, one or more patterns of coordinated sensor data across each base pod and the one or more distributed modules; generate a framework using at least one of the attributes of each base pod and each distributed module and the one or more patterns of coordinated sensor data; and generate at least one metric describing the array of aggregate sensor data based on the framework.

In some implementations, the base pod is configured to compare each pattern of coordinated sensor data to the recording file; and generate an indicator based on the comparison. In some implementations, the base pod is configured to perform dynamic time warping to at least one aggregate sensor data of the array of aggregate sensor data; and compare each dynamic time warped data to the recording file; and generate an indicator based on the comparison.

In some implementations, the system further includes a peripheral device in communication with the base pod, the peripheral device configured to receive an aggregate of the sensor data, compare the aggregate of the sensor data to an Xpression library having one or more Xpressions, and determine when a match is made between the aggregate of the sensor data and the one or more Xpressions.

In some implementations, the base pod is wirelessly connected to the one or more distributed modules. In some implementations, the base pod is physically connected to the one or more distributed modules. In some implementations, the base pod further includes memory configured to store the tacit motion.

In some implementations, at least one of the one or more sensors is configured to provide a biological attribute of the wearer.

In an exemplary embodiment, a method for generating a recording file defining a tacit motion includes receiving, by one or more distributed modules, sensor data from one or more sensors distributed at distinct locations relative to a moving body; generating, by each distributed module, an aggregate sensor data from the sensor data; communicating, by each distributed module, the aggregate sensor data to a base pod; receiving, by the base pod, an array of aggregate sensor data from the one or more distributed modules; generating a header defining an attribute of each distributed module and allowing for compiling of the array of aggregate sensor data; and generating a recording file having the header and the array of aggregate sensor data.

In an exemplary embodiment, a method for generating an Xpression associated with a tacit motion includes receiving an array of aggregate sensor data from at least one base pod and at least one distributed module, and at least one of attribute of each base pod and each distributed module; identifying, for each aggregate sensor data contemporaneously within the array of aggregate sensor data, one or more patterns of coordinated sensor data across each base pod and the one or more distributed modules; generating a framework using at least one of the attributes of each base pod and each distributed module and the one or more patterns of coordinated sensor data; and determining at least one metric describing the array of aggregate sensor data based on the framework.

In an exemplary embodiment, a method for identifying an Xpression within a recording file includes receiving a recording file having an array of aggregate sensor data and at least one attribute of a base pod and a distributed module; receiving at least one Xpression signature having at least one metric defining one or more patterns of coordinated sensor data across the base pod and the distributed module; and determining a match between the array of aggregate sensor data and the least one metric based on the at least one attribute of the base pod and the distributed module.

### BRIEF DESCRIPTION OF THE DRAWINGS

The accompanying drawings, which are incorporated in and constitute a part of the specification, illustrate one or more embodiments and, together with the description, explain these embodiments. The accompanying drawings have not necessarily been drawn to scale. Any values dimensions illustrated in the accompanying graphs and figures are for illustration purposes only and may or may not represent actual or preferred values or dimensions. Where applicable, some or all features may not be illustrated to assist in the description of underlying features. In the drawings:

FIG. 1A is a drawing of a modular tacit motion encoder system including a base pod connected to at least one distributed module, and a peripheral device in communication with the base pod according to an exemplary embodiment;

FIG. 1B is a drawing of a distributed tacit motion encoding system including a centrally located base pod in communication with a number of distributed modules positioned at distinct locations relative to a body and/or joint according to an exemplary embodiment;

FIG. 2A is a drawing of a wearable garment system having a centrally located base pod and a number of distributed modules positioned at distinct locations relative to a body and/or joint according to an exemplary embodiment;

FIG. 2B is a drawing of the wearable garment system fashioned as an arm sleeve according to an exemplary embodiment;

FIG. 2C is a drawing of the wearable garment system fashioned as a knee sleeve according to an exemplary embodiment;

FIGS. 2D-2H are drawings of the wearable garment system fashioned as a strap according to an exemplary embodiment;

FIG. 21 is a drawing of the wearable garment system fashioned as a glove according to an exemplary embodiment;

FIGS. 3A-C are block diagrams of connectivity of tacit motion encoding systems according to exemplary embodiments;

FIG. 4 is a flow diagram for a method of generating a recording file from sensor data provided by the base pod and the one or more distributed modules according to an exemplary embodiment;

FIG. 5A is a flow diagram for a method of generating an Xpression signature from a recording file according to an exemplary embodiment;

FIG. 5B is a flow diagram for a method of locating one or more base and/or distributed modules for precisely locating sensors with respect to the framework or body according to an exemplary embodiment;

FIG. 6 is a flow diagram for a method of identifying an Xpression within a recording file according to an exemplary embodiment;

FIG. 7A is drawing of an Xpression defined as a pattern in a static state of coordinated sensor data across the base pod and the one or more distributed modules according to an exemplary embodiment;

FIG. 7B is drawing of an Xpression defined as a pattern in a dynamic motion of the coordinated sensor data across the base pod and the one or more distributed modules according to an exemplary embodiment;

FIG. 8A is a representation of a recording file having a header and array of aggregate sensor data according to an exemplary embodiment;

FIG. 8B is a representation of the header mapping the array of aggregate sensor data to a body frame according to an exemplary embodiment;

FIGS. 9A-9B are a representation of dynamic time warping to a recording file according to an exemplary embodiment;

FIG. 10 is a representation of a recording file compared at a lower level according to an exemplary embodiment;

FIG. 11A is a representation of a recording file compared for consistency within a user according to an exemplary embodiment;

FIG. 11B is a representation of a recording file compared for consistency between users according to an exemplary embodiment;

FIG. 12 is a representation of a system for exchanging Xpressions between users according to an exemplary embodiment;

FIG. 13A is an illustration of a mobile app configured to operate on a peripheral device displaying a menu of app functions including creating and exchanging Xpressions between users according to an exemplary embodiment;

FIG. 13B is a screenshot of connecting a sensor/distributer module according to an example;

FIG. 13C is a screenshot of a day of Xpression activities according to an example;

FIG. 13D is a screenshot of a day of Xpression activities including a status of connected sensors according to an example;

FIG. 14A is a screenshot of a user's trends over time attempting Xpressions including progress scores, ranks, calories burned according to an example;

FIG. 14B is a screenshot of a user's competed Xpressions according to an example;

FIG. 14C is a screenshot of examples of Xpressions for volleyball according to an example;

FIG. 14D is a screenshot of a video of performing an Xpression spiking a volleyball according to an example;

FIG. 15A is a screenshot of a mobile app for authorizing uploads of recorded Xpressions according to an example;

FIG. 15B is a screenshot of a marketplace including Xpressions for purchase according to an example;

FIG. 15C is a screenshot of a marketplace including sensors/distributed modules for purchase according to an example;

FIG. 15D is a screenshot of a marketplace including Xpressions for purchase organized by activity according to an example;

FIG. 16A is a screenshot of an Xpression comparison according to an example;

FIG. 16B is a screenshot of a history of attempted Xpressions according to an example;

FIG. 17A is a screenshot of a recording of an Xpression according to an example;

FIG. 17B is a screenshot of a user's library of recorded Xpressions according to an example;

FIG. 18A is a screenshot of a database links for connecting to other users according to an example;

FIG. 18B is a screenshot of a database links for connecting to celebrity users according to an example;

FIG. 19A is a screenshot of details of a celebrity user's Xpressions according to an example;

FIG. 19B is a screenshot of reviews of a celebrity user's Xpressions according to an example;

FIG. 20A is a screenshot of activities of a first user profile according to an example;

FIG. 20B is a screenshot of activities of a second user profile according to an example;

FIG. 20C is a screenshot of other user profiles the second user profile is following according to an example; and

FIG. 20D is a screenshot of followers of the second user profile according to an example.

### DETAILED DESCRIPTION OF ILLUSTRATIVE EMBODIMENTS

The description set forth below in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

It must be noted that, as used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper,” “lower,” “interior,” “exterior,” “inner,” “outer,” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “about,” “proximate,” “minor variation,” and similar terms generally refer to ranges that include the identified value within a margin of 20%, 10% or preferably 5% in certain embodiments, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

A system and method for encoding, tracking, and comparing movements of individuals is provided that can enhance ability of individuals and trainers to monitor, communicate, compare, and instruct tacit motions in a digital form of body language. As used herein, a tacit motion can be any movement and/or pose associated with a body, an appendage relative to the body and/or relative to three-dimensional (3D) space, as well as associated attributes to the movement such as timing, force, and derivative motion attributes thereof. Examples of derivative motion attributes include bioelectric signals from physiological activity producing the movement. In some implementations, a tacit motion can be a pose held in a relaxed state or an active state. For example, a person standing still within a metro car who has their muscles relaxed may not be ready for a sudden movement of the metro car. In contrast, the same person tensing their muscles in anticipation of a movement from the metro car will be prepared to counteract the external movement.

Other examples of tacit motions include static positions and/or states such as a deliberate pose taken, a sequence of static positions and/or states, and a dynamic motion. In some implementations, a tacit motion can include any work protected by a copyright, registered copyright and/or a registered trademark including a regular image or photograph, a digital image, a fictional character, a celebrity, an emblem, a logo, a mascot, an illustration, a pictorial, a graphic, a video, a gif, as well as works not in image form such as sculptural works, which include two- and three-dimensional works of fine, graphic, and applied art. A tacit motion can include a pose and/or movement protected by a commercial license. In some implementations, a tacit motion can include virtual/augmented reality content. Further discussion of tacit motions is described in relation to FIGS. 7A and 7B below.

Tacit motions and associated attributes are encoded and captured digitally as an “Xpression.” In an aspect, an Xpression is a fuzzy description of a motion in temporal and spatial domain, with one or more fuzzy metrics. This fuzzy description can be derived from actual recordings of the sensors, and for different people with different body geometry translates to different limb movements and flexions. For instance, in a jumping jack the relative movement of each limb with respect to the other limbs matters, at the same time range of motion and intervals between segments of movement are important. Additionally, for some motions, end spatial and/or temporal metrics or features are the main objective. In an example, a pitcher releases a baseball at a certain angle and speed of arm including a twist of the wrist. In another example, a jumping jack can be encoded to an Xpression signature. When an observer sees the movement, without checking physical details of motion such as acceleration/deceleration, speed, etc. they can recognize the movement as a jumping jack. The following sequence of movements can define a jumping jack: at rest position, arms are stretched open, torso is in still position, and legs are closed, then arms move upward, legs open wide, after a brief interval, arms and legs move in opposite directions until the rest position is met again. The pattern may repeat.

In an example, an Xpression signature metric can define aspects of a “correct” jumping jack, fingertips need to touch over the head. This overall fuzzy description translates to different angles and orientations for different bodies. For each recorded impression having the physical data from the sensors, the metric can be checked against, scored or ranked (e.g. distance between left and right hand fingertips can be mathematically calculated, the lower the distance the better). An Xpression, therefore, is a mathematical description of temporal-spatial relationship of each limb, thus sensor data, in a motion, with or without defined features, independent of an actual recorded data. The temporal-spatial relation, thresholds and features can be defined manually or derived by automatically from training on repetitive data.

In an exemplary embodiment, a wearable tacit motion encoding system is provided for recording, transcribing, and influencing body motion or Xpressions of one or more wearers. In an aspect, the tacit motion system utilizes a network of sensors to detect attributes of body motion. The system is modular to distribute and to simplify processing of sensor data. In some implementations, the tacit motion encoding system can be configured to enhance performance of a wearer by providing feedback on consistency and comparative metrics to one or more Xpressions.

In some implementations, the tacit motion encoding system can be configured to notify a wearer of an imminent injury by monitoring predetermined thresholds. In an exemplary embodiment, the tacit motion encoding system can be configured to provide injury prevention feedback by monitoring a form and/or gait of the wearer by alerting the wearer and/or a trainer of improper movements.

In an exemplary embodiment, the tacit motion encoding system can be expanded to provide finer motion detection. Modularity of the tacit motion encoding system allows for additional distributed modules to form an expanded framework and provide additional granularity and nuances of tacit motions.

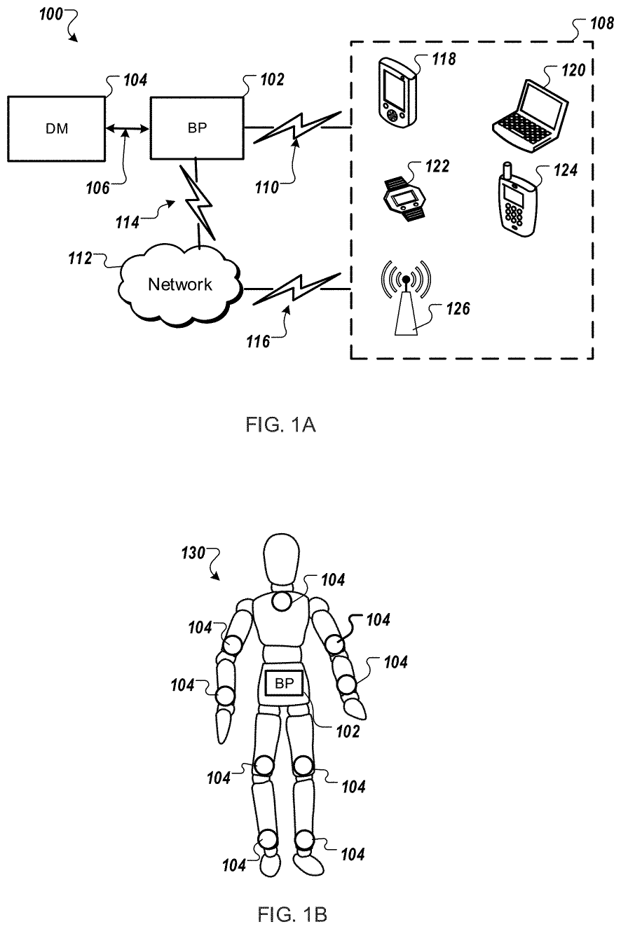

FIG. 1A is a block diagram of a modular tacit motion encoding system including a base pod 102, at least one distributed module 104, and a peripheral device 108 in communication with the base pod 102 according to an exemplary embodiment. Examples of the peripheral device 108 can include a personal electronic 118, a laptop 120 or tablet, a smart wearable device 122, a mobile device 124, and any other remote processing device 126. Portions of the tacit motion encoding system can be in communication with one another with wired and/or wireless connections. In an example, the base pod 102 can be in communication with the at least one distributed module 104 in several ways. In an example, the base pod 102 can be connected to the at least one distributed module 104 by a wire 106. In this case, data as well as power can be communicated between the base pod 102 and each distributed module 104. In another example, the base pod 102 can be wirelessly connected to the at least one distributed module 104. Similarly, the base pod 102 can be in communication with the peripheral device 108 by either a wireless or physical connection 110. In an example, the base pod 102 can be in communication with the peripheral device 108 indirectly through a network 112. In this case, the base pod 102 can be in communication with the network 112 by either a wireless or physical connection 114. Similarly, the peripheral device 108 can be in communication with the network 112 by either a wireless or physical connection 116.

In some implementations, short range wireless communication is provided through Bluetooth wireless communication technology. In other embodiments, Ultra-Wide Band (UWB) or ZigBee wireless communications may be used. The type of wireless communication technology that is used for the implementations described herein can be based on various factors that can include battery life, data usage, security and/or line-of-sight restrictions, and other concerns. In some embodiments, ZigBee or Bluetooth wireless communications may be used in applications where link security is prioritized. In other embodiments where frequency interference is a concern, Bluetooth or UWB communications may be used since both technologies use adaptive frequency hopping to avoid channel collision. In embodiments where a total of frequency channels is prioritized, Bluetooth wireless communications may be used.

In some implementations, a varying distance in-between connected portions of the tacit motion encoding system can be detected and inferred. For example, when a base pod is connected to a distributed module with a conductive tracing, an impedance check of the conductive tracing or a strain gauge sensor can be used to determine a distance between the connected portions. In an aspect, methods used for detecting faults in cable transmissions (e.g., fiberoptic, coaxial) can be applied. In the case of wireless connections, detection of similarities in electromagnetic signal distortions can be used to determine an orientation and distances through triangulation. In some implementations, portions of the tacit motion encoding system can detect and/or send a signal through the skin of a wearer to determine positioning of portions of the tacit motion encoding system. For example, two electrodes from a base pod or distributed module interfacing with the skin can determine a skin conductivity which can be used to estimate a distance between the base pod and a distributed module.

In some implementations, each base pod and distributed module can include an interface portion. The interface portion in some embodiments includes a removable integrated circuit (e.g., SIM card) and/or memory component (e.g., SD, MMC, micro-SD, etc.) and/or USB controller circuitry, and/or USB2Go controller circuitry, and/or circuitry that allows the interface portion to be recognized by the Android® operating system or other operating system as may be used by the tacit motion encoding system, and/or an energy storage device such as a battery or super/ultra-capacitor (which may be in addition to an energy storage device), and/or circuitry to support a software license key manager, such that software installed on the base pod and each distributed module can be modulated, activated, unlocked, updated, or modified by circuitry and firmware or software on through the interface portion.

The tacit motion encoding system can be distributed at various locations on a wearer to capture a tacit motion. As shown in FIG. 1B, a distributed tacit motion encoding system 130 includes a centrally located base pod 102 in communication with a number of distributed modules 104 positioned at distinct locations relative to a body and/or joint.

In some implementations, a tacit motion encoding system can be used to measure limb orientation to recreate a body pose. In an example, the number of distributed modules 104 can be positioned at distinct locations to measure joint dynamics, impact force (e.g., acceleration and body mass) to the joint, as well as torque to the joint. In an example, the number of distributed modules 104 can be positioned at distinct locations to determine a symmetry of a movement. In an example, the number of distributed modules 104 can be positioned at distinct locations to determine an orientation of a limb (pronation). In an example, a distinct location can be determined by a biometric and/physiological signal from the body. In an aspect, a distinct location can be defined by a location where both a biometric signal and motion can be correlated. In an example, a distinct location can be defined by a position providing a sufficient range of motion for one or more finite states within a frame.

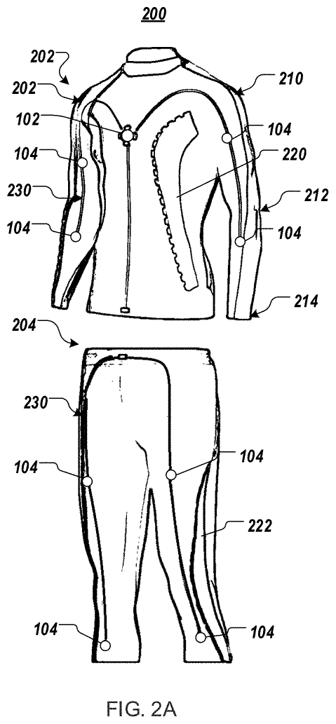

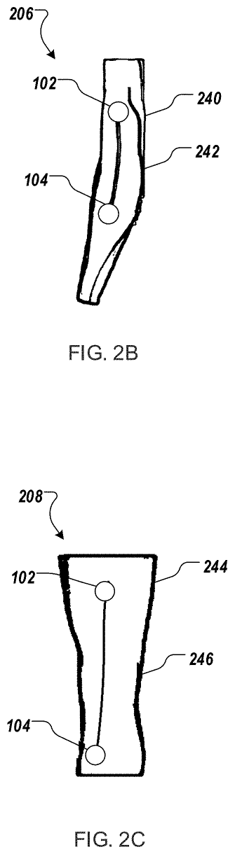

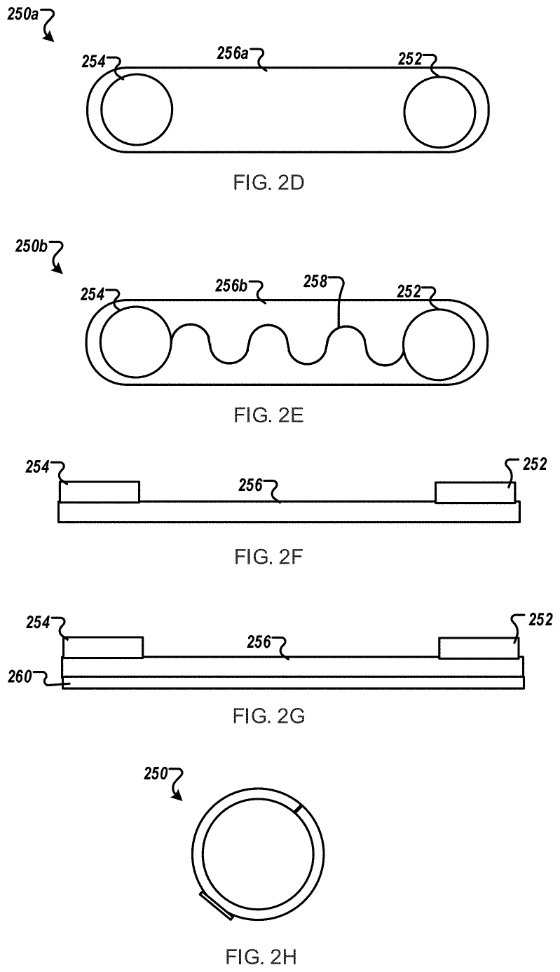

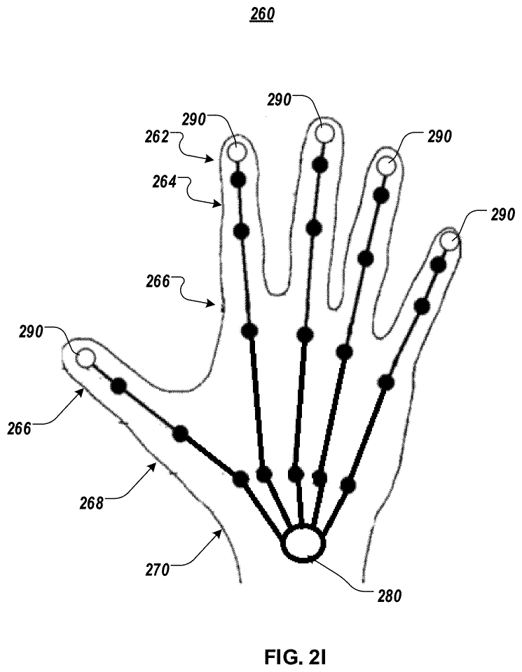

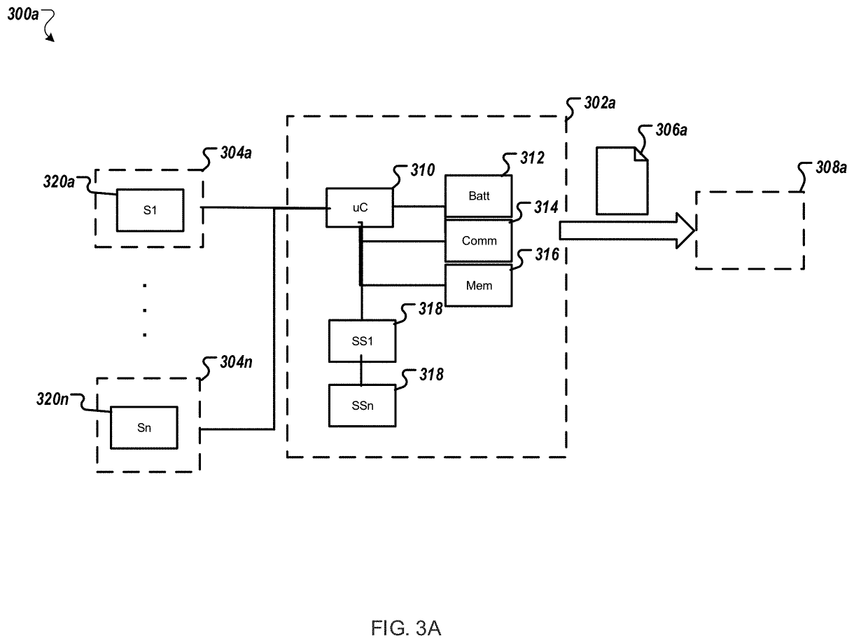

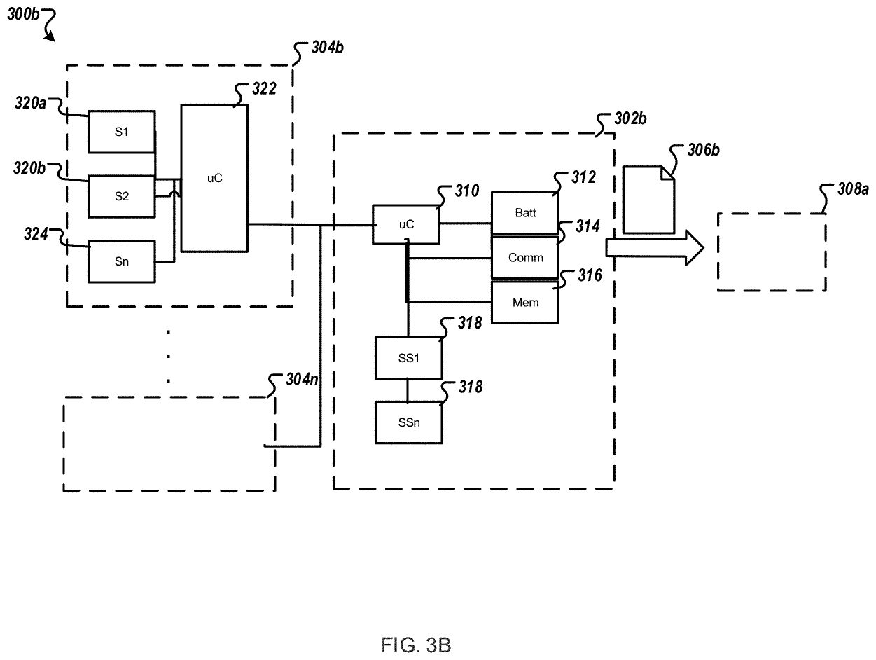

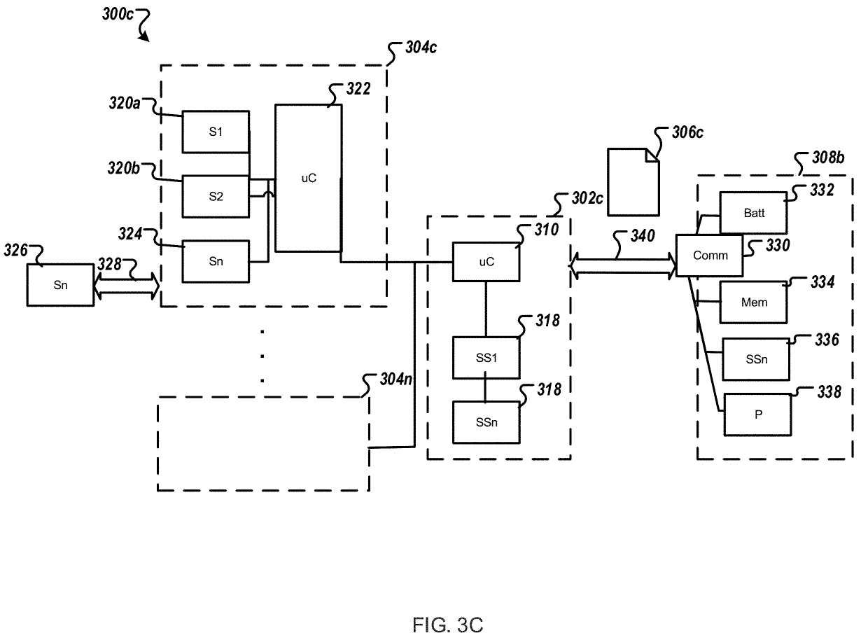

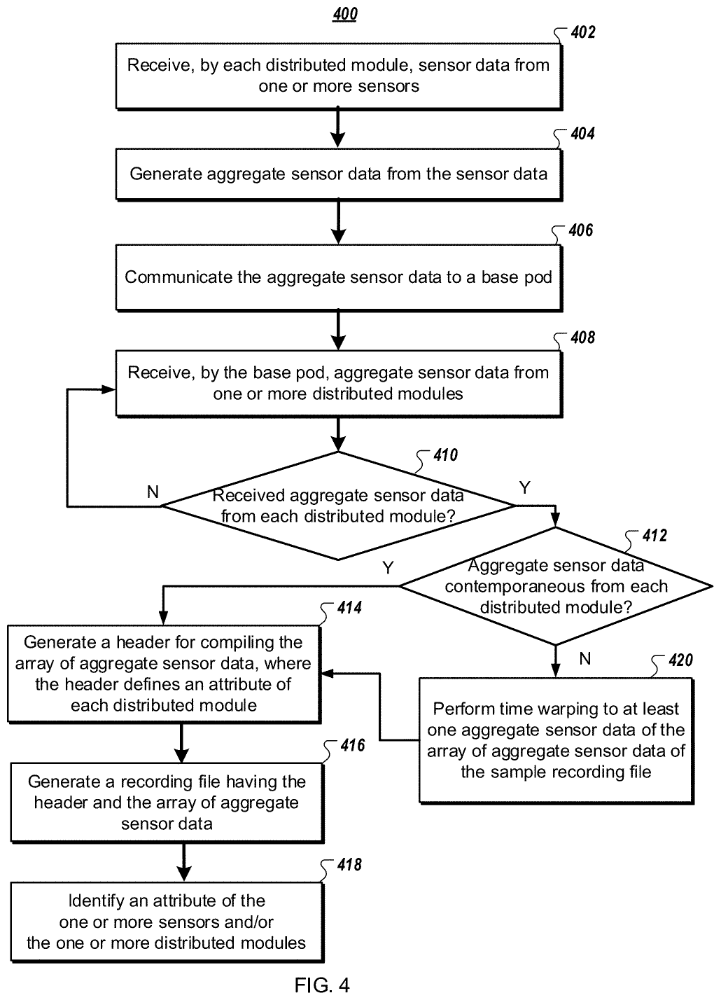

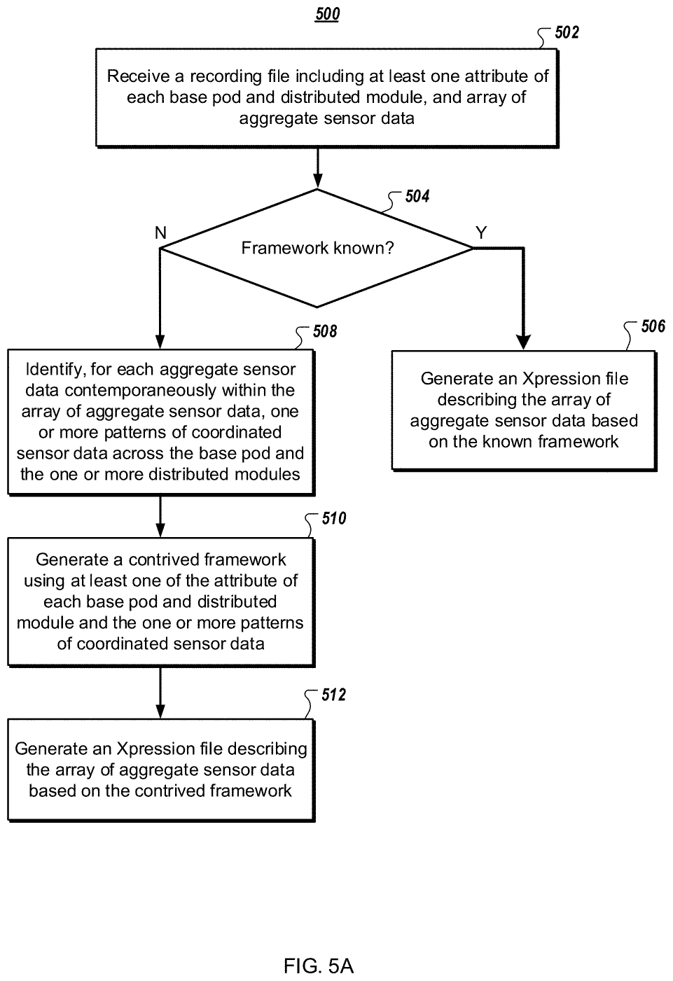

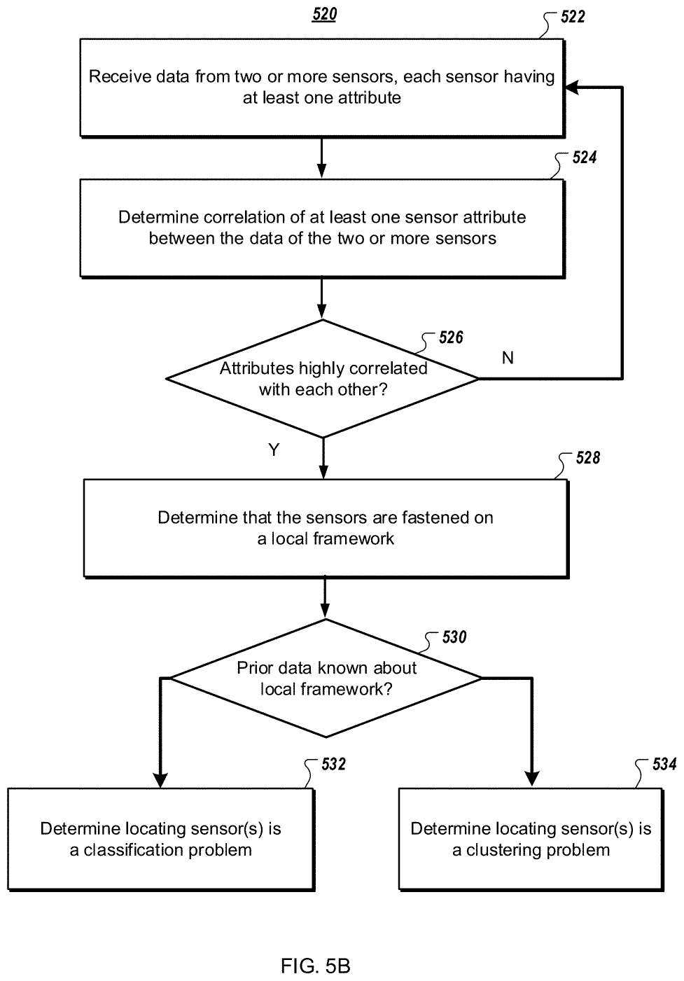

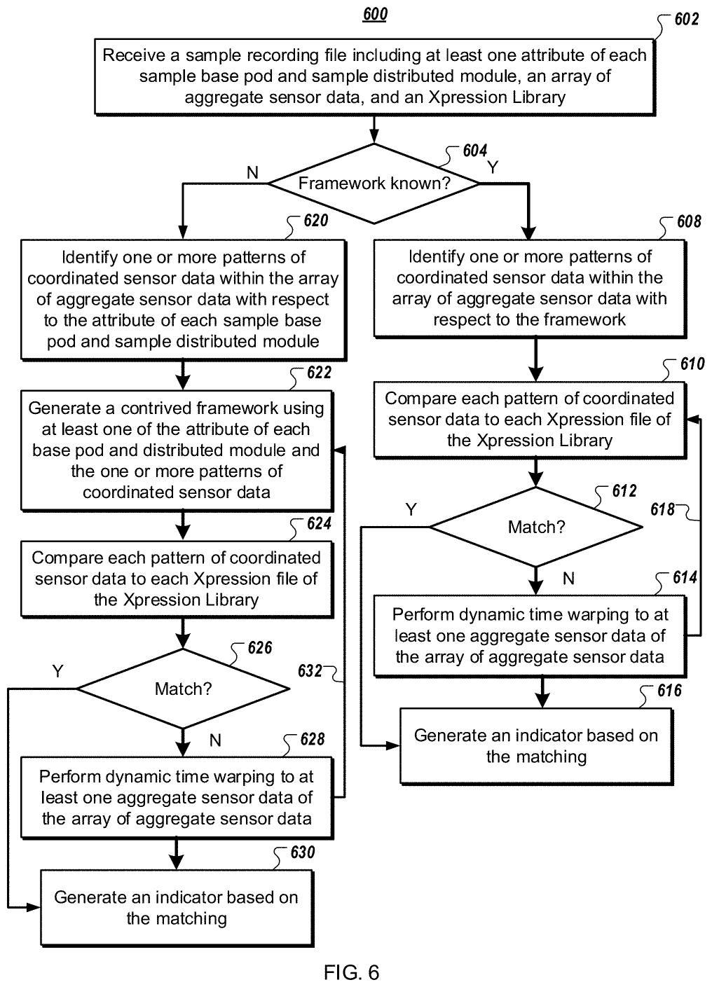

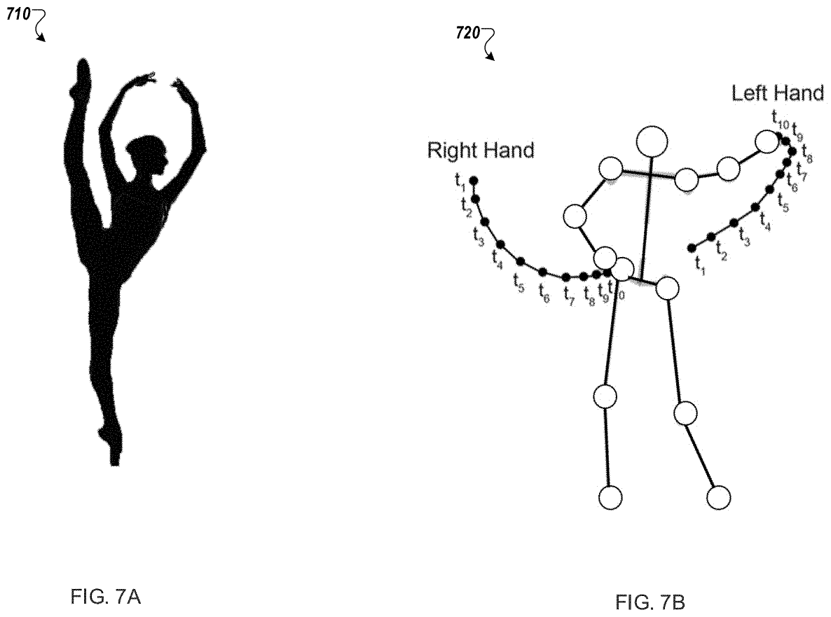

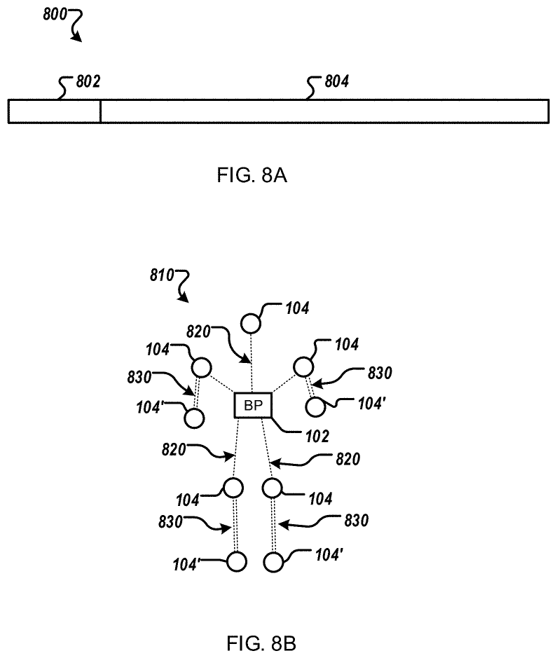

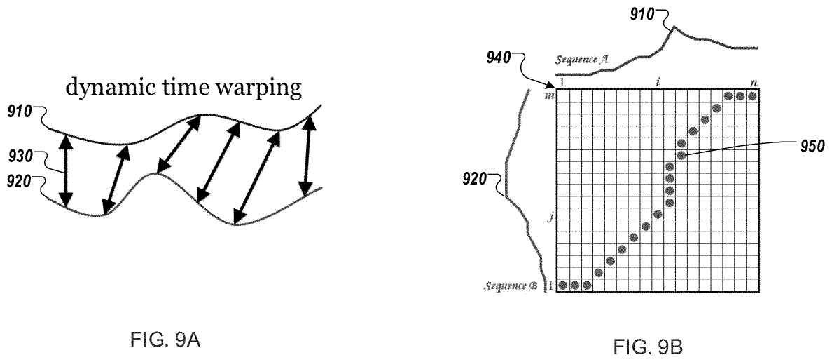

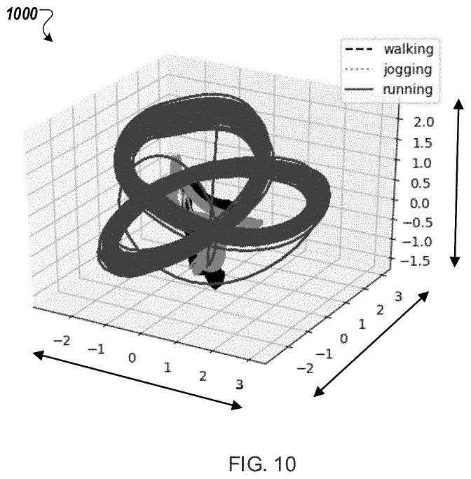

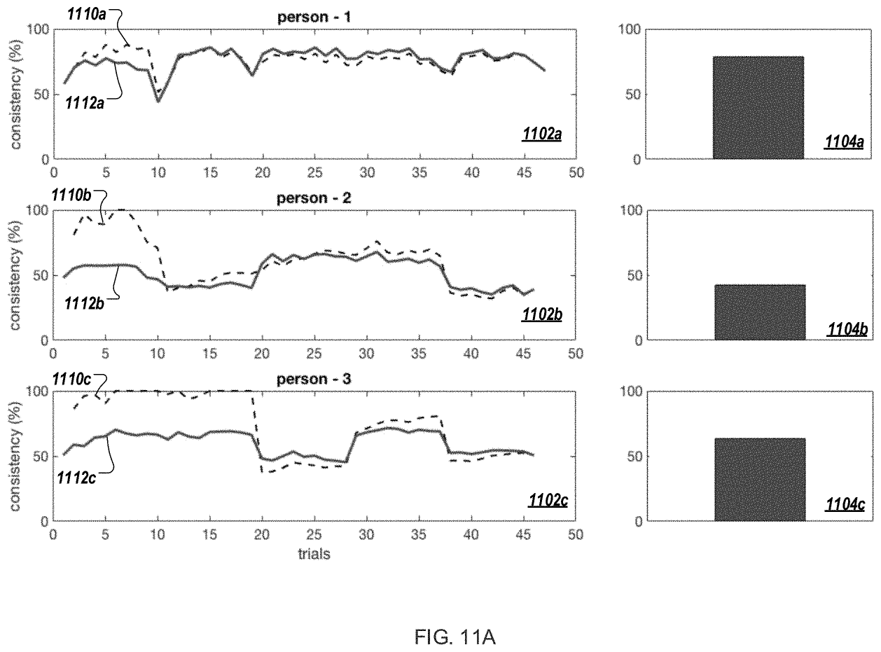

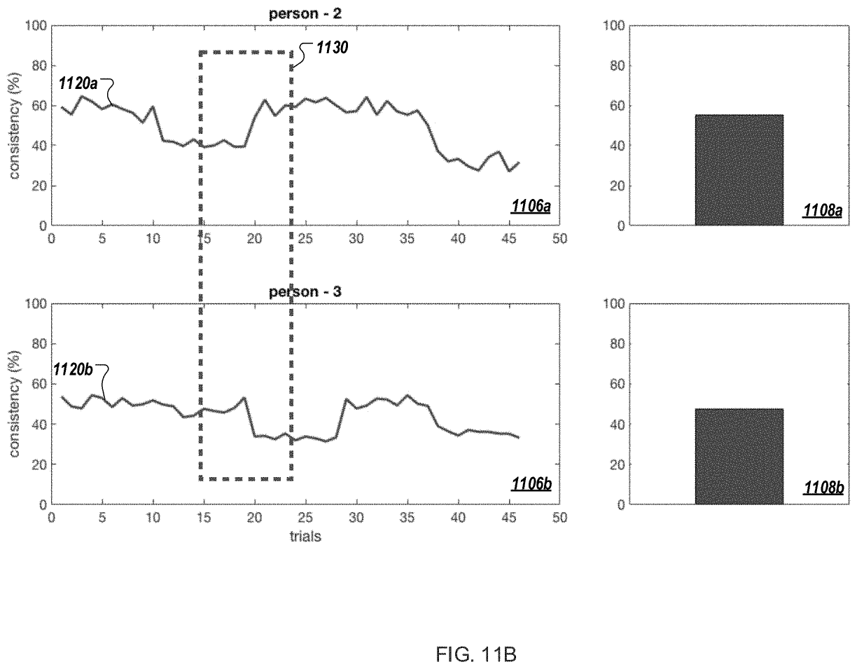

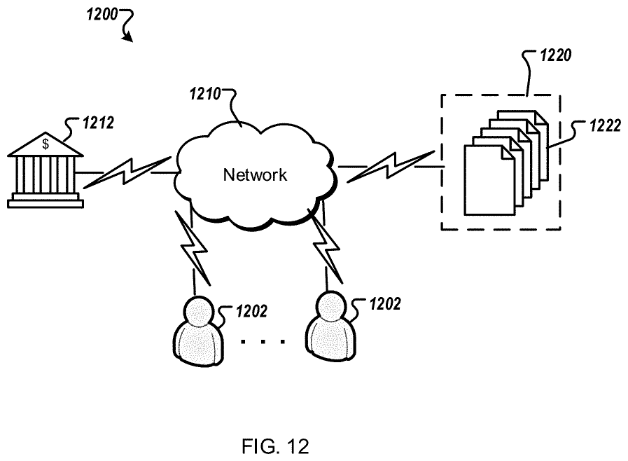

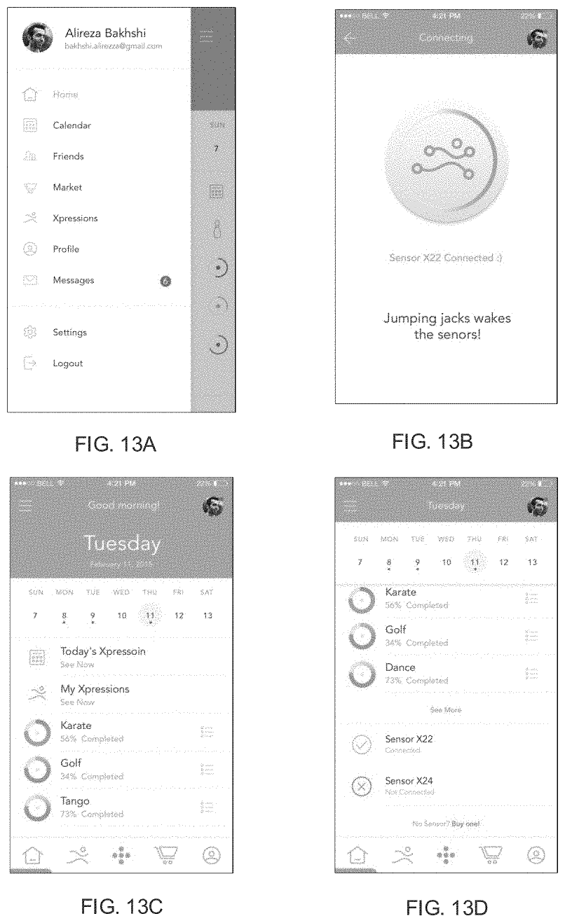

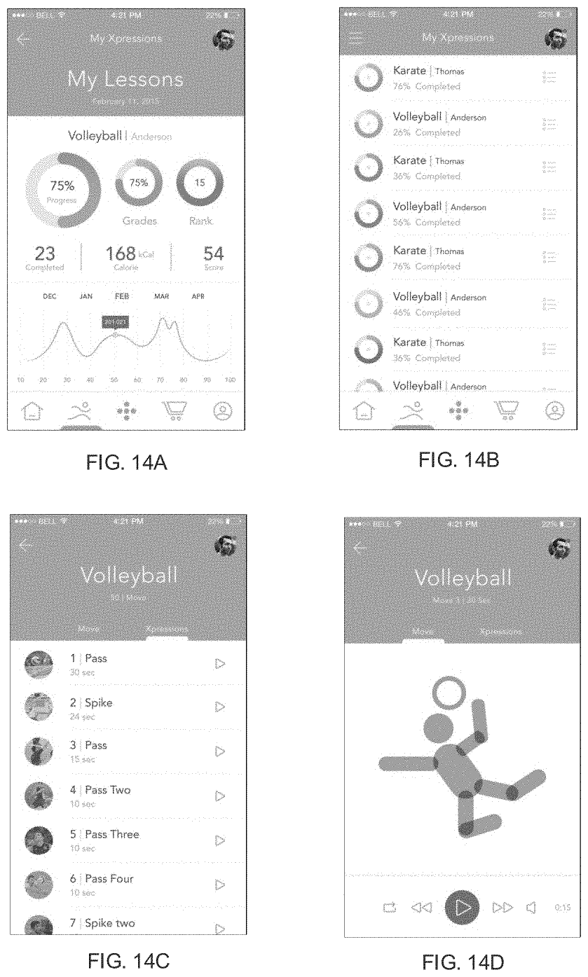

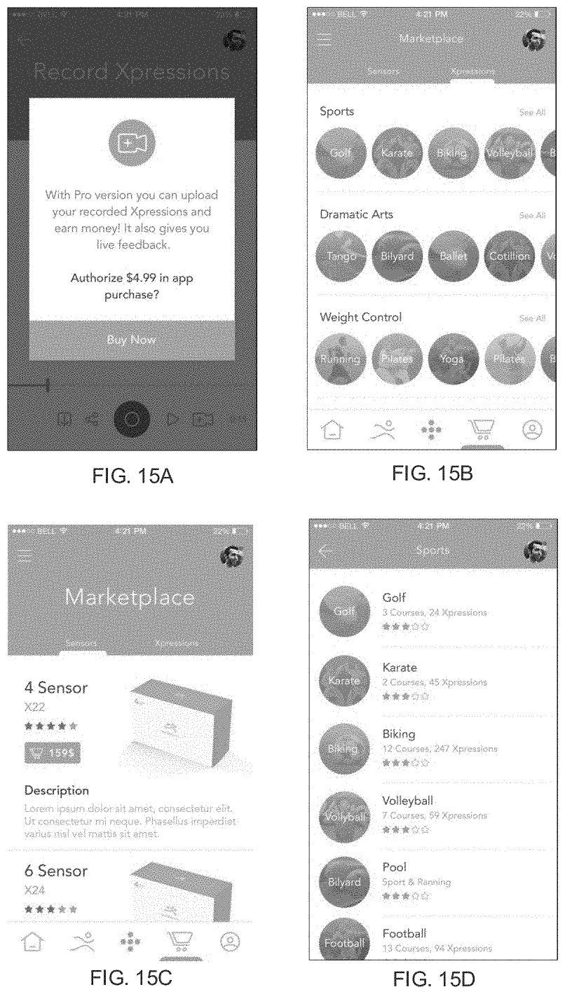

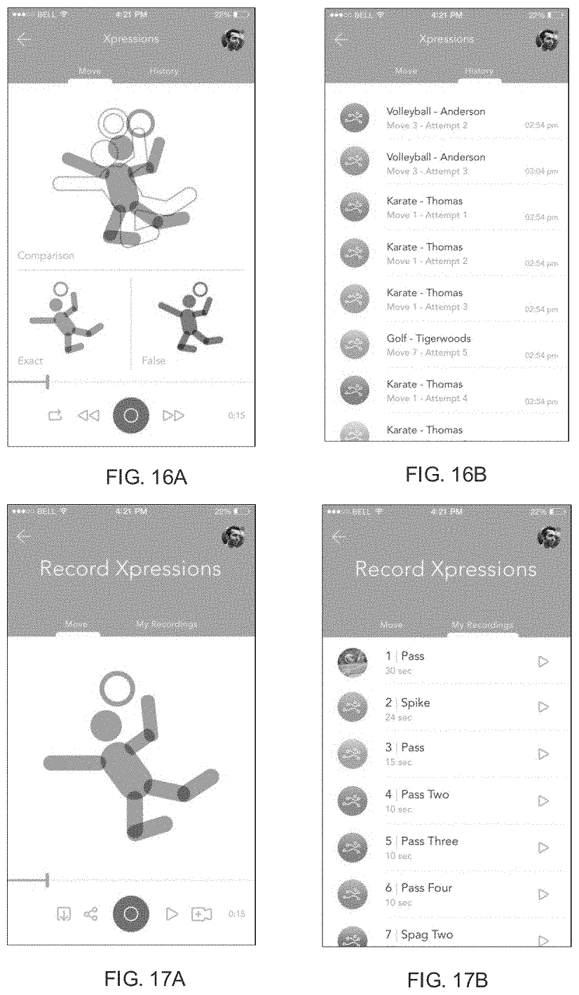

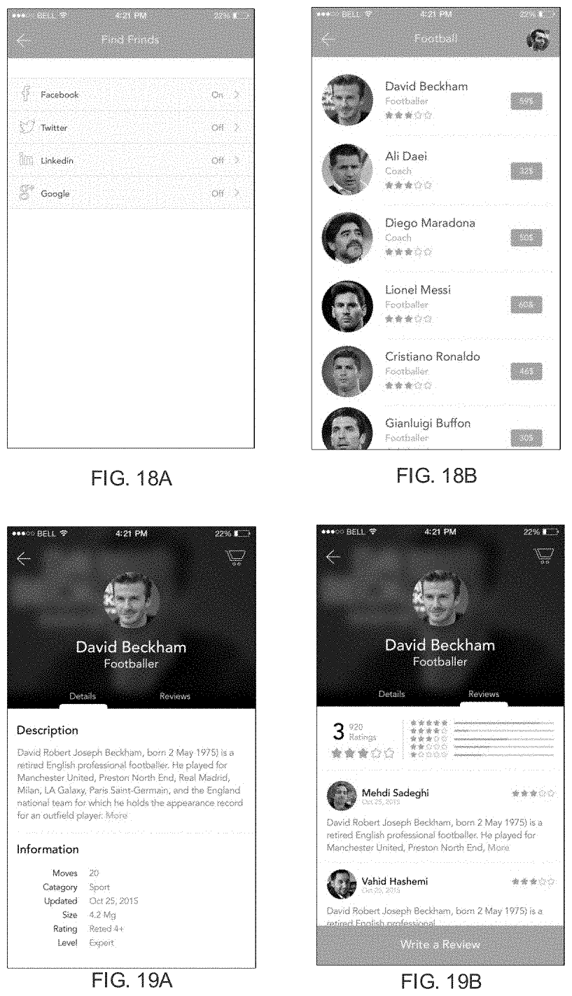

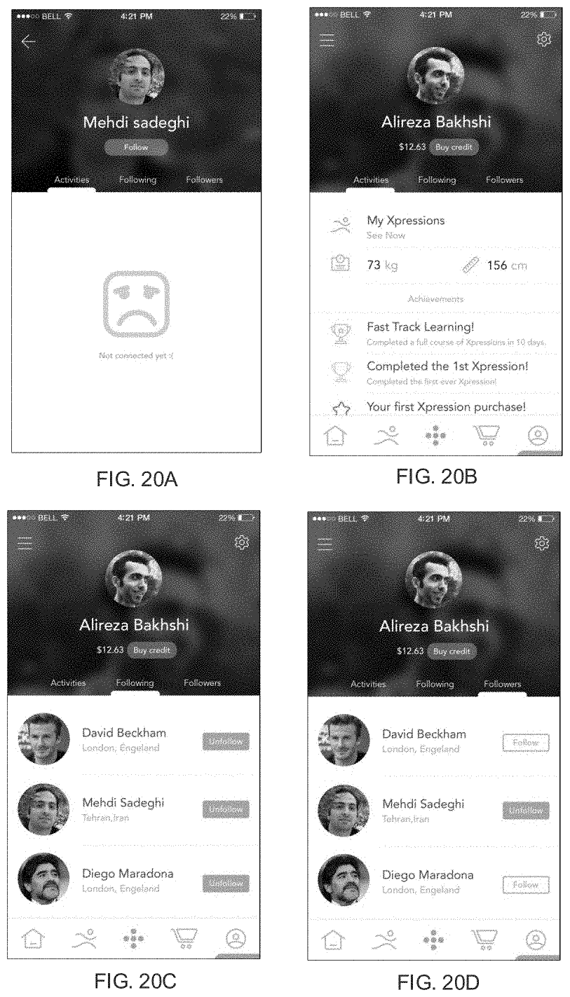

In some implementations, the distributed tacit motion encoding system 130 can include a wearable garment system 200 having the number of distributed modules 104 positioned at the distinct locations relative to a body and/or joint. Examples of wearable garment systems include but are not limited to a top 202 and bottom garment 204 (FIG. 2A), an arm sleeve 206 (FIG. 2B), a knee sleeve 208 (FIG. 2C), and an encoding glove 260 (FIG. 21). In an aspect, the wearable garment system can be configured to capture a tacit motion associated with an appendage relative to a main body and/or 3D space. In another aspect, the wearable garment system can be configured to capture an aspect of a joint such as the arm sleeve 206 (FIG. 2B) which is configured to detect attributes of an elbow joint and the knee sleeve 208 which is configured to detect attributes of a knee joint (FIG. 2C). In an example, the arm sleeve 206 includes a sleeve 240 having a base pod 102 and at least one distributed module 104, where the base pod 102 is proximal to an elbow joint 242 and the at least one distributed module 104 is distal to the elbow joint 242. In an example, the knee sleeve 208 includes a sleeve 244 having a base pod 102 and at least one distributed module 104, where the base pod 102 is proximal to a knee joint 246 and the at least one distributed module 104 is distal to the knee joint 246.

In an aspect, each wearable garment system can be configured to include a base pod and one or more distributed modules that can be expanded as needed according to an exemplary embodiment. In an exemplary embodiment, the tacit motion encoding system can be expanded to provide finer motion detection. In an example, the encoding glove 260 can provide digit movement for each finger. (FIG. 21) In an example, the encoding glove 260 can include a base pod 290 in communication with distributed modules 290 for encoding movements for the pinkie distal interphalangeal joint 262 (DIP), the proximal interphalangeal (PIP) joint 264, the metacarpophalangeal (MCP) joint 266, the thumb interphalangeal (IP) joint 268, and the encoding joint 270.

In some implementations, the wearable garment system can include a fabric and conductive tracing 230 printed on the fabric and/or sown into the fabric for connecting the base pod to the one or more distributed modules. In an aspect, the fabric can be breathable to aid comfort of the wearer. In some implementations, the wearable garment system can have portions of conductive fabric 220, 222 functioning as a sensor component, where the conductive fabric 220, 222 is in communication with the base pod and/or the one or more distributed modules. In an example, the conductive fabric 220, 222 can be made from electroactive materials and shape memory materials. Examples of electroactive materials and shape memory materials may include materials such as electroactive polymers, dielectric elastomers, and shape memory alloys such as nitinol.

In some implementations, a wearable garment system can include a base pod and one or more distributed modules positioned along an encoding strap that can be placed on and/or around any portion of the body according to an exemplary embodiment. As shown in FIG. 2D, a tacit motion encoding system 250a-b can include an encoding strap or strap 256 having a base pod 252 in communication with at least one distributed module 254 attached to the strap 256. In an example, the strap 256b can have wiring 258 connecting the base pod 252 to each distributed module 254. Alternatively, the base pod 252 can be in wireless communication with each distributed module 254.

Locations of the base pod 252 relative to the one or more distributed modules 254 and/or the wearer can be determined automatically or manually according to an exemplary embodiment. In an example, the wearer can manually enter a location and/or orientation of the strap relative to the body and/or a body extension. Examples of body extensions include prosthetics, tools, and other accessories.

In an example, the strap 256a-b can include an attachment mechanism 260 such as Velcro, a snap backing, and an adhesive. (See FIG. 2G) The attachment mechanism 260 can be used in adhering the strap 256a-b to a garment worm by the individual and/or directly on a skin of the individual. In some implementations, the strap 256a-b can rigid, semi-rigid, or a flexible wrap.

In some examples, processing of sensor data obtained by each distributed module 104, base pod 102, and peripheral device 108 can be performed by control circuitry such as a programmable logic controller (PLC) or central processing unit (CPU) that executes one or more software processes and outputs position information to other controllers and electronically-activated components. FIGS. 3A-3C provide a simplified hardware block diagram of control circuitry of a tacit motion system 300a-c. The description of the control circuitry is not meant to be limiting, and can include other components than those described herein. References to control circuitry relate to the circuitry of one or more processing circuits, which can also be referred to interchangeably as processing circuitry. The control circuitry may include a processor or central processing unit (CPU) 322, 338 that executes one or more software processes associated with the tacit motion encoding system 300a-c. Software instructions for the processes can be stored in memory 316, 334. In some examples, the memory 316, 334 can include both volatile and non-volatile memory and can store various types of data associated with executing the processes related to collecting sensor data from one or more sensors 320a-c, 324.

In some implementations, the sensor data can be stored on an internal storage medium and/or a removable storage medium of each distributed module. In some examples, the local storage medium may include a removable storage medium (e.g., a removable SIM or other removable memory of the distributed module), or a built-in memory location of the distributed module.

In some implementations, a tacit motion encoding system 300a-c can be configured to divide and/or distribute processing of sensor data. In an aspect, the division and distribution of the processing of sensor data can be done for power savings, computational efficiency, and to offload host computational capacity. In an aspect, the division and distribution of the processing of sensor data improves modularity.

In some implementations, rather than or in addition to encrypting the sensor data, the distributed modules may reformat the sensor data into a preferred data format.

In an exemplary embodiment, a tacit motion encoding system 300a-c can include a base pod 302a-c (102) in communication with one or more distributed modules 304a-n (104), where the base pod 302a-c is configured to receive processed sensor data from the one or more distributed modules 304a-n and to output an avatar 306a-c representing a digitally-captured tacit motion of a wearer. The avatar 306a-c can be stored locally on the memory 316, 334 and communicated to a peripheral device 308a-b (108) is a single upload or by streaming.

In an exemplary embodiment, the avatar 306a-c can be formed by assigning aggregate sensor data to a known rigid body (e.g., established body frame). In some implementations, a portion of the avatar 306a-c can be stored in a digital media standard format such as Biovision hierarchical data format (bvh). In another exemplary embodiment, the avatar 306a-c can be formed by machine learning and constraint optimization for assigning the aggregate sensor data to a generated phantom (e.g., generated frame). In an example, a distance between any one of a sensor, one or more distributed modules, and the base pod can be estimated and/or sensed for generating or determining a generated frame. In an example, an impedance check similar to transmission lines can be done to determine the distance. In another example, an integrated strain gauge sensor in a flexible strap can be sensed.

In some implementations, the avatar 306a-c includes a header and a data section that includes sensor data. In an example, the avatar 306a-c can have a header for defining metrics for the tacit motion. In an example, the header can describe a hierarchy of the sensors 320a-c and each distributed module 304a-n and initial pose of their framework (e.g., skeleton). In an example, the header can be generated by the processor on the base pod. Alternatively, the header can be generated by the peripheral device 108. In some implementations, the header can define an accuracy of the tacit motion. In an aspect, the header can include where the Xpression was collected from in a sequence of the avatar 306a-c. In some implementations, the header can include one or more control Xpresssions for starting, stopping, editing, or modifying a metric or attribute of the recorded sequence. For example, a motion of raising a person's arms in an ‘X’ configuration can stop a recording session.

In an exemplary embodiment, at least one of the one or more sensors is configured to provide a biological attribute of the wearer. In some implementations, the avatar 306a-c can include sensor data providing biological attributes of the wearer as sensed by one or more biosensors 324. Examples of biological attributes of the wearer can be skin temperature, skin conductance, Electromyography (EMG) signals, as well as other measurands that can be coordinated with a movement and/or physiological status. A cold muscle/ligament is known to not stretch as much as a warmed up muscle/ligament. In an example, the tacit motion encoding system can record a skin temperature to correlate the skin temperature with a range of motion.

In some implementations, as shown in FIG. 3C, a tacit motion encoding system 300c can be configured to communicate the sensor data to the peripheral device 308b for processing of the sensor data. In this case, the base pod 302c can be configured to utilize resources on the peripheral device 308b.

In some implementations, the tacit motion encoding system 300a-c can be configured to generate an alert to the wearer and/or the trainer of improper movements. In an example, the base pod 302c, the one or more distributed modules 304a-n, and/or the peripheral device 308a-b can include one or more of an alarm, a haptic vibrator, and an LED configured to provide feedback to the wearer. In an example, the alert can be generated by the base pod 302c and/or a respective one or more distributed modules 304a-n corresponding to a feedback location. For instance, when a feedback includes that an arm should be positioned higher, a respective distributed module 304a-n positioned on the arm can generate an alert for notifying the wearer that their hand is not in proper position. The alert can be modulated based on a magnitude and type of difference with a proper positioning. For example, an audio tone can play at a sound intensity level, a tone frequency, and/or a burst frequency to provide feedback that the wearer is either closer or farther from matching the proper positioning. In some implementations, the tacit motion encoding system can provide feedback (e.g. alert) to the wearer for positioning of each distributed module. In an example, the tacit motion encoding system can alert that the wearer has achieved an Xpression indicated by a commercial license.

In some implementations, each distributed module 304a-n can include one or more of one or more inertial measurement unit (IMU) sensors 320a-c configured to sense an IMU movement, one or more biosensors 324 for sensing a biological attribute of the wearer, and a microprocessor 322 for processing data and communicating the processed data to the base pod 302b-c and/or another distributed module 304a-n. In an aspect, each distributed module 304a-n can be configured to sense one or more parameters associated with the distinct location, to compute and/or synthesize the one or more parameters and to communicate information based on the computation or synthesis to the base pod 102, 302a. In some implementations, each distributed module 304a-n can have one or more inertial measurement unit (IMU) sensors 320a-c configured to sense an IMU movement. In some implementations, each distributed module 304a-n can have a microprocessor 322 configured to process sensor data from the one or more IMU sensors 320a-c and to communicate the processed data to the base pod 302b-c. (FIG. 3A) In an example, the microcontroller 322 is configured to output angular data based on the sensor data.

In some implementations, a first IMU sensor of the one or more IMU sensors 320a-c can be an accelerometer configured to detect an acceleration in an x, y, and z direction. In some implementations, a second IMU sensor of the one or more IMU sensors 320a-c can be a gyroscope configured to detect a gyration and/or a rate of rotation in an x, y, and z direction. In some implementations, a third IMU sensor of the one or more IMU sensors 320a-c can be a magnetometer configured to detect a gravitational field in an z direction. In an aspect, a combination of one or more of the one or more IMU sensors 320a-c can be configured to detect rotation angles (e.g., pitch, roll and yaw) of the distributed module 304a-n. In some examples, the sensors 320a-c may also include other types of sensors, such as sensors associated with determining an impedance, capacitance, vibration, etc. In an exemplary embodiment, a system of sensors can be arranged for providing high accuracy detection of a position with respect to an external frame of reference (e.g. earth) from sensor data including acceleration, rate of rotation, GPS (or GNSS or similar) data, and optionally magnetometer data.

In some implementations, as shown in FIG. 3B, the one or more distributed modules 304a-n includes one or more biosensors 324 for sensing a biological attribute of the wearer. Examples of biological attributes of the wearer include heart rate, oxygen concentration, temperature, perspiration, EMG. In some implementations, the microprocessor 322 can be configured to process data from the one or more biosensors 324 and communicate the processed data to the base pod 302b-c.

In an aspect, each distributed module 304a-n can include a microphone configured to detect a tacit sound. In an example, the microphone can be a bone-conducting microphone. Examples of a tacit sound can include cardiovascular sounds such as heart beats and breathing, skeletal sounds such as bone rubbing, cracking, popping, and sounds generated from movement such as tapping, as well as hard/soft landings.

In an aspect, a base pod 302a-c is responsible for collecting, parsing, and consolidating sensor data from each distributed module 304a-n. In some implementations, a portion and/or the whole wearable garment system can be considered as one of the one or more distributed modules. In some implementations, the base pod 302a-c can be configured to store the collected sensor data in memory and can communicate the collected sensor data to the peripheral device 308. As shown in FIG. 3B, the base pod 302a can have one or more of a microcontroller 310, a power source 312, a communication module 314, a memory 316, and one or more sensors 318. The microcontroller 310 can be configured to receive sensor data from each distributed module 304a-n and to process the sensor data into the avatar 306a-c. The microcontroller 310 can be configured to transmit power from the power source 312 and instructions to each distributed module 304a-n. The power source 312 can be a Lithium Ion or Lithium Polymer battery or other similar energy storage device. The power source 312 may include a charging apparatus such as an induction charging apparatus for connection-free charging of the battery or other similar energy storage device to which it may be attached or otherwise electrically coupled. The power source 312 may include one or more energy harvesting devices and kinetic charging mechanisms to charge the power source, maintain power source charge, and slow depletion thereof.

Examples of instructions include a setting and/or a modification of any one of a sensitivity of the one or more sensors, a sampling rate for recording sensor data, a measurement range, identification of which sensors to use, and one or more thresholds.

In an example, the base pod can determine that a particular distributed module is positioned differently, (e.g., rotated, positioned more distal, positioned more proximal) as compared to another distributed module or a previous recording. In an example, the base pod can modulate individual sensor data and/or the aggregate sensor data from the respective distributed module to compensate and account for the difference in positioning. In another example, the base pod can send an instruction to the distributed module to compensate and account for the difference in positioning. In some implementations, the tacit motion encoding system can provide feedback (e.g. alert) to the wearer for positioning of each distributed module.

In an example, the instructions can be received by the communication module 308 and/or from the memory 316. In some implementations, the microcontroller 310 can be further configured to receive data from one or more auxiliary sensors 326.

In an exemplary embodiment, each of the one or more distributed modules 304a-n can be configured to output a raw stream of sensor data. In some implementations, each of the one or more distributed modules 304a-n can be configured to output processed data of the sensor data by the microcontroller 322.

In some implementations, as shown in FIG. 3C, the tacit motion encoding system 300c can be configured to use one or more components and functionalities of a peripheral device 308b such as a battery 332, a memory 334, a communication module 330, a processor 338, and one or more sensors 336. In an example, the tacit motion encoding system 300c can include a base pod 302c having a microcontroller 310 in communication with the one or more distributed modules 304a-n. In this case, the tacit motion encoding system 300c can utilize the peripheral device's 308b battery 332 to power the base pod 302c and/or the one or more distributed modules 304a-n. In an example, the tacit motion encoding system 300c can utilize the peripheral device's 308b memory 334 for storing the sensor data. In an example, the tacit motion encoding system 300c can utilize the peripheral device's 308b processor 338 for processing the sensor data. In an aspect, the peripheral device 308b can be connected to the base pod 302c with a connector. The connector can be wired or an inductor coil for wireless power and data transfer. The communication module 330 can be used to facilitate data and energy transfer 340 between the peripheral device 308b and the base pod 302c.

In an example, at a high level, when a wearer performs a tacit motion, the sensors are configured to detect one or more changes associated with the motion, the microcontroller of each distributed module receives sensor data from each sensor, determines an aggregate sensor data (e.g. an angle determined from the sensor data), and communicates the aggregate sensor data to the base pod. The base pod compiles each aggregate sensor data from each distributed module and updates at least one metric in the header. In some implementations, the base pod is configured to store an attribute of the sensors, and/or the aggregate sensor data, and the header in a transferable file that is streamed to the peripheral device and/or stored in memory of the base pod. In some implementations, the transferable file can include an entire recording or a buffer of most recent time series of sensor data and spatial-temporal data. In an aspect, an Xpression is a most concise part of the transferable file and can include metrics defining movements protected by a commercial license.

Turning to FIG. 4, a flow chart illustrates in example method 400 for generating a recording file from sensor data provided by the base pod and the one or more distributed modules according to an exemplary embodiment. In an exemplary embodiment, the method 400 for generating a recording file can include steps of receiving, by each distributed module, sensor data from one or more sensors (402), generating an aggregate sensor data (e.g. angle(t)) from the sensor data (404), and communicating the aggregate sensor data to a base pod (406), receiving, by the base pod, aggregate sensor data from the one or more distributed modules (408), and determining if the aggregate sensor data was received from each distributed module (410). When the aggregate sensor data was not received from each distributed module (N), the method 400 returns to the previous step 408. When the aggregate sensor data was received from each distributed module (Y), the method 400 determines if the aggregate sensor data is contemporaneous from each distributed module. When the aggregate sensor data is contemporaneous from each distributed module (Y), the method includes generating a header for compiling the array of aggregate sensor data, where the header defines an attribute of each distributed module (414), and generating a recording file having the header and the array of aggregate sensor data (416).

Returning to step 412, when the aggregate sensor data is not contemporaneous from each distributed module (N), the method includes performing time warping to at least one aggregate sensor data of the array of aggregate sensor data of the sample recording file (420). After performing time warping, the method 400 continues to step 414.

In some implementations, the method 400 includes identifying an attribute of the one or more sensors and/or the one or more distributed modules (418). In some implementations, generating aggregate sensor data from the sensor data (404) includes calculating an angle of movement based on sensor data from the one or more sensors 318, 320a-c, 324, 336. In some implementations, generating aggregate sensor data from the sensor data (404) includes receiving biodata from the one or more biosensors 324. In some implementations, generating aggregate sensor data from the sensor data (404) includes encoding a snippet of the sensor data.

The step of receiving, by each distributed module, sensor data from one or more sensors (402) can be done in several ways. As shown in FIG. 3A, a distributed module 304a may have only a single sensor 320a where sensor data is directly passed on to the base pod 302a. As shown in FIG. 3B, a distributed module 304b may have multiple sensors 320a-b including different sensor types (324) where sensor data is aggregated by the microcontroller 322 to be passed on to the base pod 302b. As shown in FIG. 3C, a distributed module 304c may have multiple sensors 320a-b including different sensor types (324) and receive data from one or more auxiliary sensors 326, where sensor data is aggregated by the microcontroller 322 to be passed on to the base pod 302b.

The step of communicating the aggregate sensor data to a base pod (406) can be done in several ways. In an example, the microcontroller 322 can communicate aggregate sensor data to the base pod 302b by streaming through wired and/or wireless connections. In another example, the distributed module 304a may store sensor data on a local memory which is read by the base pod at a later time. While the flow diagram illustrates an ordering of steps or blocks of the method 400, it can be understood that the various steps and processes associated with the method 400 can be performed in any order, in series, or in parallel.

Turning to FIG. 5A, a flow chart illustrates in example method 500 for generating an Xpression signature from a recording file according to an exemplary embodiment. In an exemplary embodiment, the method 500 for generating an Xpression signature can include steps of receiving the recording file including at least one attribute of each base pod and distributed module, and the array of aggregate sensor data (502), determining if a framework is known (504). In an example, attributes of each base pod and distributed module can be stored in a header of the file. When a framework is known (Y), the method 500 includes generating an Xpression signature describing the array of aggregate sensor data based on the known framework.

Examples of known frameworks include the body frame. When a framework is not known (N), the method 500 includes identifying, for each aggregate sensor data contemporaneously within the array of aggregate sensor data, one or more patterns of coordinated sensor data across the base pod and the one or more distributed modules (508), generating a contrived framework using at least one of the attribute of each base pod and distributed module and the one or more patterns of coordinated sensor data (510), and generating an Xpression signature describing the array of aggregate sensor data based on the contrived framework (512).

In some implementations, the known frameworks can be described in choreography or dance notations representing known symbolic representations of human dance movement and form. Examples of dance notations include Labanotation, Kinetography Laban, Benesh Movement Notation, Eshkol-Wachman Movement Notation, Motif Notation, and DanceWriting. In an example, Labanotation is a detailed description of movement so it may be reproduced exactly as it was performed or conceived. In an example, the Motif Notation depicts the most important elements or the essential aspects of a movement sequence. In an example, Benesh Movement Notation can use abstract symbols based on figurative representations of the human body to plot a position of a dancer as seen from behind, as if the dancer is superimposed on a staff that extends from the top of a dancer's head down to their feet, including additional segments of the staff coincides with the head, shoulders, waist, knees and feet, etc. In an example, transcription of Labanotation can include direction (e.g., front back, diagonal) symbols, level (e.g., low, middle, high) symbols, body weight support indicators, holding indicators.

Examples of attributes of each base pod and distributed module used for defining metrics include sensor types, sensor identification, sensor data information (e.g., range, variance, noise). In an example, sensor information from a period of a recording can be classified, coordinated, and used to contrive a framework varying on a magnitude of the sensor information.

In some implementations, identifying one or more patterns of coordinated sensor data (508) includes clustering of sensor data by converting the sensor data into a lower level space or principle component analysis.

In an aspect, the method 500 can be performed by a processor on the base pod and/or the peripheral device. While the flow diagram illustrates an ordering of steps or blocks of the method 500, it can be understood that the various steps and processes associated with the method 500 can be performed in any order, in series, or in parallel.

In some implementations, the system can be used to perform a method 520 of locating one or more base and/or distributed modules for precisely locating sensors with respect to the framework or body. Examples of precisely locating sensors can include at least one of a sensor position relative to a portion of a framework, sensor orientation, heading, and reliability. As shown in FIG. 5B, in some implementations, the method includes receiving data from two or more sensors, each sensor having at least one attribute (522), determining a correlation of at least one sensor attribute between the data of the two or more sensors (524), and determining if the attributes highly correlated with each other (526). When the attributes are not highly correlated with each other (No), return to step 522. When the attributes are highly correlated with each other (Yes), determine that the sensors are fastened on a local framework (528). Examples of sensors are fastened on a local framework include being on a same limb or an accessory in coordination with the limb. When the sensors are determined to be fastened on a local framework, the method 520 can further determine if prior information of the type of recordings from different body locations are known (530). When prior information of the type of recordings from different body locations are known (Yes), the method determines locating is a classification problem (532). When prior information of the type of recordings from different body locations are not known (No), the method determines locating is a clustering problem (534). In an example, clustering can be used to first categorize prior knowledge or data into sub-groups such as limb, posture, etc. In an example, classification or nearest-neighbor can be used to classify an unknown sensor into one of these sub-groups.

In some implementations, the classification problem can be divided into a semi-locating method and a fully-locating method. In an example, the semi-locating method can be used for fine tuning wearable suits to different body forms and sizes, where an approximate location for sensors are known from the geometry of the suit. In another example, the semi-locating method can be used for adjusting sensors in action improves quality of captured motion. In an example, the full-locating method can be used for predicting a location of a stamp-on/strap-on sensors with respect to the 3D body, when no information is given about individual sensors. In another example, the full-locating method can be used for locating sensors on accessories with relation to body, such as where the accessory is in relation to the body.

In an example, a method 900 for dynamic time warping (DTW method) is shown pictorially in FIGS. 9A-9B. FIG. 9A shows a first sequence 910, a second sequence 920, and a mapping 930 of the first sequence 910 and the second sequence 920. FIG. 9B shows a dynamic programming plot 940 for comparing the first sequence 910 and the second sequence 920. In an aspect, the DTW method 900 measures similarity between time series sequences that vary in length. In an example, the DTW method 900 can be used to assess similarity of locomotion coming from different people. While a gait speed may vary between different people, each gait speed can be similarly classified as walking. The DTW method 900 searches for an optimal match between sequences. When the sequences are similar, the DTW method 900 returns comparatively a small number in comparison to when the sequences are not similar. In an example, similarity of the sequences is demonstrated in a dynamic programming plot 940 shown in FIG. 9B.

In an aspect, the dynamic programming plot 940 demonstrates a process of estimating a distance between two sequences 910, 920. The dynamic programming plot 940 includes a pairwise distance matrix having N×M dimensions, where N is a length of a first sequence 910 and M is a length of a second sequence 920. Each value within the pairwise distance matrix represents a distance (i.e. Euclidean distance) between the first sequence 910 at a time point t_n(i) and the second sequence 920 at a time point t_m(j).

The DTW method 900 includes generating, within the pairwise distance matrix, a correlation line 950 which represents a shortest distance to reach to each end of the sequences.

For example, assume the following pairwise distance matrix:

$$\begin{matrix}.8 & .5 & .3 & .1 \\ .7 & .4 & .1 & .3 \\ .4 & .1 & .5 & .2 \\ .4 & .1 & .6 & 0 \\ .1 & .3 & .1 & 0\end{matrix}$$

As shown in this very simplified example, the 0.1 values form a contemporaneous path along that extends from a beginning and end of each sequence. In some implementations, for longer sequences, to reduce search time, the DTW method 900 can include one or more restrictions on a portion of the pairwise distance matrix to reduce computations while maintaining performance. Examples, of restrictions can include a type or nature of the sequences, and how far apart can they can be stretched in time and still be similar.

Turning to FIG. 6, a flow chart illustrates in example method 600 for identifying Xpressions within a recording file according to an exemplary embodiment. In an exemplary embodiment, the method 600 for identifying an Xpression within a recording file can include steps of receiving a sample recording file including at least one attribute of each sample base pod and sample distributed module, an array of aggregate sensor data, and an Xpression Library (602) and determining if a framework is known (604). In an example, determining if a framework is known (604), the method 600 further includes determining when a framework describing relativity of the array of aggregate sensor data is known. In an example, the attributes of each sample base pod and sample distributed module can be received in a header of the recording file.

When the framework is known (Y), the method 600 includes identifying one or more patterns of coordinated sensor data within the array of aggregate sensor data with respect to the framework (608), comparing each pattern of coordinated sensor data to each Xpression signature of the Xpression Library (610), and determining if there is a match between the sample recording file and an Xpression signature of the Xpression Library (612). When a match is found (Y), the method 600 includes generating an indicator based on the matching (616). In this case, the indicator can identify the Xpression signature found matching along with any comparative analytics. When a match is not found (N), the method 600 includes performing dynamic time warping to at least one aggregate sensor data of the array of aggregate sensor data (614) and returning (618) to step 610 and comparing each pattern of coordinated sensor data, modified by the dynamic time warping, to each Xpression signature of the Xpression Library. In some implementations, when a match is not found (N), the method 600 includes generating an indicator based on the matching (616). In this case, the indicator can identify a portion of a closest Xpression signature found matching along with any comparative analytics.

Returning to step 604, when the framework is not known (N), the method 600 includes steps of identifying one or more patterns of coordinated sensor data within the array of aggregate sensor data with respect to the attribute of each sample base pod and sample distributed module (620), generating a contrived framework using at least one of the attribute of each base pod and distributed module and the one or more patterns of coordinated sensor data (622), comparing each pattern of coordinated sensor data to each Xpression signature of the Xpression Library (624), and determining if there is a match between the sample recording file and an Xpression signature of the Xpression Library (626). When a match is found (Y), the method 600 includes generating an indicator based on the matching (630). In this case, the indicator can identify the Xpression signature found matching along with any comparative analytics such as identifying the contrived framework and attributes used. When a match is not found (N), the method 600 includes performing dynamic time warping to at least one aggregate sensor data of the array of aggregate sensor data (628) and returning (632) to step 622 and generating a different contrived framework using at least one of the attribute of each base pod and distributed module and the one or more patterns of coordinated sensor data, modified by the dynamic time warping. In some implementations, when a match is not found (N), the method 600 includes generating an indicator based on the matching (630). In this case, the indicator can identify a portion of a closest Xpression signature found matching along with any comparative analytics for the contrived framework and any attribute of each sample base pod and sample distributed module.

In an aspect, the method 600 can be performed by a processor on the base pod and/or the peripheral device. While the flow diagram illustrates an ordering of steps or blocks of the method 600, it can be understood that the various steps and processes associated with the method 600 can be performed in any order, in series, or in parallel.

Examples of pattern include a static state 710, holding of a static state, and a dynamic motion 720. In some implementations, the static state 710 can be considered as coordinated sensor data at a moment in time across the base pod and the one or more distributed modules. (See FIG. 7A) For example, a static motion 710 can be when one or more sensors are used to detect a stance such as a ballerina's stance. In an example, the holding of the static state 712 can be detected for isometric strength training where a time is recorded of the wearer in the stance. In some implementations, the pattern is a dynamic motion 720 of the coordinated sensor data across the base pod and the one or more distributed modules. (See FIG. 7B) For example, the dynamic motion 720 can be when one or more sensors detect a motion of a left arm and a right arm. In an example, the dynamic motion 720 can require that other sensors are substantially in the same position. In an aspect, the dynamic motion 720 can require an intensity/speed, a rhythm, a fluidity, and an abruptness of a motion. For example, in a dance move mimicking the “moon walk” a fluidity of exchanges of foot motion is required. In contrast, a “robotic” dance requires short bursts of discrete motions which are aimed to augment rigidity of a stance.

FIG. 8A is a representation of a recording file 800 having a header 802 and array of aggregate sensor data 804 according to an exemplary embodiment. FIG. 8B is a representation of the header mapping the array of aggregate sensor data to a body frame 810 according to an exemplary embodiment. In an example, the header can define a first connection 820 between a base pod 102 and a first distributed module 104, and a second connection 830 between the first distributed module 104 and a second distributed module 104′. While these are exemplary, further connections and configurations can be established by the header.

In some implementations, the body frame has a known or predetermined geometry. In this case, the header can include information related to the geometry or constraints. In some implementations, the body frame has an unknown or undetermined geometry or constraints. In this case, the wearer can be prompted to perform a movement such as a jumping jack to determine locations and attributes of each sensor, distributed module, and base pod. In some implementations, a second/third order differential of comparisons from sensor data from the base pod and at least one distributed module can be used for determining a frame structure of interest. In an example, when the sensors are presenting noise, artifacts, and/or oscillating or vibrating in a relative, common or coordinated manner, an effective thickness of a plane can be determined. In an example, the effective thickness of a plane can determine placement of the base pod or a distributed module on a front or back side of an otherwise 2D body. These common artifacts from each sensor can be extracted to provide a parameter for building the frame structure of interest.

In some implementations, sequences and array of aggregate sensor data can be transformed to a lower level space or dimension to aid in machine learning and automatic characterization. In an example, characterizations of a sequence can reveal distinct movement patterns of walking and running including under pronation and over pronation of the movement. In some implementations, a method 1000 for performing low-dimensional dynamics (LDD) is shown pictorially in FIG. 10. In an aspect, the LDD method 1000 can be used for visualization and generalization of sequences for reducing noise and redundancy in data. Sequence data originally in high dimensions can be difficult to visualize and understand. Visualization of a sequence includes a step of projecting the sequence down to 2D and/or 3D such that the sequence can be plotted. For visualization of sequences, the LDD method 1000 can be performed by implementing data visualization methods including principal component analysis, factor analysis (FA), and linear dynamic system (LDS).

For generalization of sequences, the LDD method 1000 can be performed by identifying repeated patterns of activity from the same person. For example, a wearer can perform a number of iterations of a tacit motion (e.g., jumping jacks) while trying their best to replicate the exact same motions. Although the iterations may appear to be similar by viewing the movements, recordings of the iterations by the tacit motion encoding system can be quite different. This difference between iterations can be identified as noise by the LDD method 1000. In another case, similar trends identified within the iterations are considered redundancies. In an aspect, the LDD method 1000 can purify the distances provided by the DTW method 900. In an aspect, the LDD method 1000 can be used for modifying the sequences. In an example, modifications include scaling and rotating the data. In an example, the LDD method 1000 can be used for comparing scaled metrics such as magnetometer data with accelerometer data and rotations of the sensors from one person wearing them to another.

Tacit motions can be compared in several ways for determining consistency. In an example, consistency of a movement can be determined by comparing a number of trials of performing one or more Xpressions. In an example, the number of trials can be a cumulative number or a moving average of a predetermined number of trials.

In an aspect, a consistency attribute can be generated by comparing data of a given user over a number of iterations in a causal (i.e. realtime) or non-causal way (i.e. offline). In an example, a P number of movements can be compared in a non-casual way (i.e. offline method) where each movement will be compared to P−1 number of movements resulting in P−1 number of DTW values. An average of the P number of movements can be reported for each of the P separate movements. In another example, in the causal way, when a first movement is received, there is nothing to compare with, so the comparison is skipped. When a second movement is received, a comparison can only be done to the first movement. In this case, the comparison can be done by averaging the movements and a comparison value can be reported. When a third and additional movements are received, a comparison can be done to determine an average and/or median value of all of the movements.

Turning to FIG. 11A, representations of recording files compared for consistency of repeating one or more Xpressions within a user 1102a-c from actual data are shown according to an exemplary embodiment. In an example, a user's performance 1110a-c can be tracked for a number of trials for repeating an Xpression. For example, a first user 1102a can repeat one or more Xpressions and record a consistency 1110a for attempting the one or more Xpressions over a number of trials. The consistency 1110a curve reflects a median value representing the first user's 1102a consistency of matching each Xpression and/or an initial trial. Upon completion of the number of trials, a median consistency 1112a curve is determined and compared to all previous trials. The median consistency can be determined from the consistency 1110a over the entire number of trials or a subset of the number of trials.

In some implementations, an overall consistency 1104a-c can be determined throughout a number of trials. In an example, the overall consistency 1104a-c can be generated based on averaging a user's median consistency 1112a over the entire number of trials or a subset of the number of trials. In an example, the overall consistency 1104a-c can be provided as feedback to the user in real-time and/or as a summary after a session of trials. The consistency can vary during different portions of the number of trials. In an example, the consistency can be high during a first portion of the number of trials and low during another portion of the number of trials. In an aspect, an amount of fatigue can be detected by comparing the consistency throughout different portions of the number of trials. As illustrated within bar charts 1104a-c of FIG. 11A, the first user 1102a overall was more consistent than a second user 1102b and a third user 1102c.

Representation of consistency between two or more users can be done similarly as above with causal and/or non-causal methods, and realtime and/or offline methods, including hybrids thereof. In this case, movements are received from at least two different users, but averages from more than two users can also be used. For example, a P number of movements can be received from a first person and a Q number of movements can be received from a second person. In the case of offline comparisons, each movement of the first person will be compared to all movements of the second person, thus having Q number of separate DTW comparisons. An average consistency can be reported based on the DTW comparisons. In the case of realtime comparisons, movements can be received simultaneously based on a buffer of the data received. In an example, two people are performing a dance or a game where each attempt to perform similar movements. An average consistency can be reported based on the realtime DTW comparisons.

Turning to FIG. 11B, a representation of consistency between two or more users 1106a-b performing one or more Xpressions from actual data is shown according to an exemplary embodiment. Comparison curves 1120a-b represent a comparison between the median consistency 1112a of a first wearer to the median consistency 1112b-c for each wearer respectively. In this example, the median consistency 1112a from the first user 1102a repeating the one or more Xpressions in FIG. 11A was compared to that of the median consistency 1112b from the second user 1102b and the median consistency 1112c from the third user 1102c.

Indicator 1130 identifies a portion of the trials where the users were asked to modify their movements to be more and less similar to a reference user. In the case of the second user 1102b (curve 1120a), their movements became more consistent with movements of the first user 1102a, showing a positive trend. In the case of the third user 1102c (curve 1120b), their movements became less consistent with movements of the first user 1102a, showing a negative trend. An overall score 1108a-b can be generated based on an average of the comparison of the median consistency 1112a from the first user 1102a to the median consistency 1112b from the second user 1102b and the median consistency 1112c from the third user 1102c, respectively. In an example, the overall score 1108a-b can be used as feedback to each respective wearer.

FIG. 12 is an illustration of a system 1200 for exchanging Xpressions between users including an Xpressions library according to an exemplary embodiment. The system 1200 can include a network 1210 for connecting users 1202, a remote computing system or server 1220 hosting an Xpressions library 1222 according to an example.

In an aspect, the Xpressions library can be configured for secured exchanging of Xpressions and sensor data by and between a number of wearers and trainers. In some implementations, a portion of an Xpression and/or the Xpressions library can be stored in the memory 316, 334, the peripheral device 108, and/or the remote computing system or server 1220 in communication with the network 112, 1210. The Xpressions library can be operated by a user to upload and/or download Xpressions to the memory 316, 334, the peripheral device 108, or Xpressions library including both licensed and non-licensed digitally captured tacit motions. The Xpressions library additionally provides digital rights management, upload and download monitoring functions, user account management, and messaging functions. The Xpressions library manages both licensed and non-licensed images for purposes of obeying licensing laws when uploading/downloading the Xpressions into the local memory of the tacit motion encoding system. Licensed digital Xpressions, such as copyrighted Xpressions including trademarked poses/movements having licensing terms and conditions for usage can be leased/purchased from one or more online sources. Users generally access the online environment in the Xpressions library to search, select, edit, and purchase Xpressions.

In some implementations, the Xpressions library can be filtered to display Xpressions based on compatible or available hardware to the user. For example, when the user only has a minimal or partial system, the Xpressions can be shown that are available to capabilities of their system. In some implementations, the Xpressions library can be agnostic to the hardware, where multiple different types of hardware can be used to provide an Xpression.

In an exemplary embodiment, the system can include a mobile app configured to operate on the peripheral device for creating and exchanging Xpressions between users, as well as for monetizing the Xpressions. Turning to FIGS. 13A-D, the mobile app can display a menu of app functions including creating and exchanging Xpressions between users (FIG. 13A) according to an exemplary embodiment. The mobile app can display connections to a sensor/distributer module and instructions for initiating a wake up cycle (FIG. 13B). The mobile app can display a day of Xpression activities (FIG. 13C) including a status of connected sensors (FIG. 13D) according to an example.

Turning to FIGS. 14A-D, the mobile app can display a user's trends over time attempting Xpressions including progress scores, ranks, calories burned (FIG. 14A) according to an example. FIG. 14B is a screenshot of a user's competed Xpressions according to an example. In an example, the mobile app can include a number of Xpressions for various volleyball moves (FIG. 14C) including a video of performing each Xpression (FIG. 14D). FIG. 14D shows a video of an Xpression spiking a volleyball according to an example.

In some implementations, the mobile app can include a marketplace for authorizing uploads of recorded Xpressions for other users to purchase (FIG. 15A), a marketplace for downloading Xpressions for purchase (FIG. 15B), and a marketplace for purchasing sensors/distributed modules/wearable garments (FIG. 15C). In an example, the marketplace can organize Xpressions for purchase by activity such as sports, dramatic arts, weight control etc. (FIGS. 15B, 15D). Examples of sports includes golf, karate, biking, volleyball, soccer. Examples of dramatic arts include dance, tango, ballet, etc. Examples of weight control includes running, lobbies, yoga, etc.

In some implementations, the mobile app can include a comparison playback of a user's trial compared to an Xpression (FIG. 16A) as well as their history of trials attempting each type of Xpression (FIG. 16B). In some implementations, the mobile app can include a recording feature for recording Xpressions (FIG. 17A) and storage for holding a user's library of recorded Xpressions (FIG. 17B). In some implementations, the mobile app can include database links for connecting to other users in their social networks (FIG. 18A), as well as to celebrity or elite users (FIG. 18B). Examples of a profile for a user, celebrity or elite user can include details, reviews, and followers (FIGS. 19A-FIG. 20C).

In an exemplary embodiment, the tacit motion encoding system can be configured for individual use, for group training, and a hybrid between both. Examples of hybrid uses include fitness training where a trainer can monitor multiple trainees during a workout, and continue to monitor activity outside the group workout. In an exemplary embodiment, the tacit motion encoding system can be configured for physical therapy and rehabilitation. In an example, the tacit motion encoding system can position distributed modules for detecting a joint force and range of motion of an appendage. Further, progress for the joint force and the range of motion of the appendage can be monitored and tracked for progress.

In an exemplary embodiment, the tacit motion encoding system can be configured for use capturing dramatic arts. In an example, an Xpression of a dramatic arts movement can be registered as a copyright. In some implementations, the tacit motion encoding system can be employed by a team of users such as a cheerleading team. At competitions, judges can use analytics provided by the tacit motion encoding system to quantify and monitor conformity of the team in their assessment of performance. In an exemplary embodiment, the tacit motion encoding system can be configured for multi-player gaming where each gamer wears a tacit motion encoding system which corresponds to a game avatar within a common game.

In an exemplary embodiment, the tacit motion encoding system can have a sampling rate tailored for a given application. For example, in a fast speed application the sampling rate can be increased to detect higher resolution movements. In an aspect, the tacit motion encoding system can have distributed modules at locations where biometrics at each location are more relevant to forming the avatar.

In some implementations, the tacit motion encoding system can be used to send a command to the peripheral device and/or another device. In an example, upon detection of an Xpression, the tacit motion encoding system can be configured to send a control signal to the peripheral device to start or stop a service on the peripheral device. In an example, upon detection of an Xpression, the tacit motion encoding system can be configured to send a control signal to remotely controlled devices including a drone, a camera, electronic vehicle such as a passenger vehicle, skateboard, utility equipment such as a crane etc. In an exemplary embodiment, the tacit motion encoding system can be configured for use in communicating instructions in a construction zone. For example, a construction worker can perform an Xpression of a safety hazard, a directions command for large equipment, etc. In an example, the Xpression can be communicated to another worker (e.g., via a display, speaker, and haptic device). In an example, the Xpression can be communicated to a display and/or traffic sign to control a status and/or a command to modify traffic (e.g., stop or go for traffic in a construction zone).

While certain embodiments have been described, these embodiments have been presented by way of example only, and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

In an exemplary embodiment, a system for communicating a tacit motion includes a base pod having memory and a communication port; one or more distributed modules, each distributed module comprising at least one sensor for sensing a tacit motion of a wearer; and where each distributed module is in communication with the communication port.

In an exemplary embodiment, at least one of the one or more sensors is configured to provide a biological attribute of the wearer.

In an exemplary embodiment, a method for generating a recording file includes receiving, by one or more distributed modules, sensor data from one or more sensors distributed at distinct locations relative to a moving body; generating, by each distributed module, an aggregate sensor data from the sensor data; communicating, by each distributed module, the aggregate sensor data to a base pod; receiving, by the base pod, an array of aggregate sensor data from the one or more distributed modules; generating a header defining an attribute of each distributed module and allowing for compiling of the array of aggregate sensor data; and generating a recording file having the header and the array of aggregate sensor data.

In an exemplary embodiment, a method for communicating a tacit motion includes generating a recording file, generating a Xpression from the recording file, and identifying one or more Xpressions within a recording file.

In an exemplary embodiment, generating the Xpression signature includes receiving at least one attribute of each base pod and distributed module, and the array of aggregate sensor data; identifying, for each aggregate sensor data contemporaneously within the array of aggregate sensor data, one or more patterns of coordinated sensor data across the base pod and the one or more distributed modules; and generating an Xpression signature describing the one or more patterns of coordinated sensor data across the base pod and the one or more distributed modules.

In an exemplary embodiment, identifying an Xpression within a recording file includes receiving a sample recording file including at least one attribute of each sample base pod and sample distributed module, the array of aggregate sensor data, and a library of Xpression signatures, where each Xpression signature describes one or more metrics defining one or more patterns of coordinated sensor data across at least one base pod and at least one distributed module; performing, based on the attribute of each sample distributed module, dynamic time warping to at least one array(t) of the array of aggregate sensor data of the sample recording file; identifying a pattern of coordinated sensor data for each aggregate sensor data within the array of aggregate sensor data of the sample recording file; comparing the one or more patterns of coordinated sensor data across the base pod and the one or more distributed modules of each Xpression signature of the library of Xpression signatures to the pattern of coordinated sensor data for each aggregate sensor data within the array of aggregate sensor data of the sample recording file; and generating an indicator for each matching Xpression within the sample recording file.

1. A wearable system for recording a tacit motion, the system comprising:
at least one base pod, each base pod having a processor and at least one first sensor in communication with the processor, the at least first sensor configured to sense a first motion of a wearer;
one or more distributed modules, each distributed module comprising at least one second sensor for sensing a second motion of the wearer, each second sensor configured to be in communication with the processor of the base pod and to be positioned at a distinct location relative to a different body part of the wearer; and
wherein each sensor is positioned at a different distinct location relative to the wearer;
wherein coordinated data from each sensor is configured to be used for detecting a tacit motion defined at least in part by the first and second motions of the wearer.

2. The system of claim 1, wherein each base pod is configured to
receive a recording file including an attribute of each base pod and each distributed module, and an array of aggregate sensor data associated with the tacit motion, one or more metrics associated with an Xpression;
identify, for each aggregate sensor data contemporaneously within the array of aggregate sensor data, one or more patterns of coordinated sensor data across each base pod and the one or more distributed modules;
generate a framework using at least one of the attributes of each base pod and each distributed module and the one or more patterns of coordinated sensor data; and
generate at least one metric describing the array of aggregate sensor data based on the framework.

3. The system of claim 1, wherein each base pod is configured to:
compare each pattern of coordinated sensor data to the recording file; and
generate an indicator based on the comparison.

4. The system of claim 1, wherein each base pod is configured to:
perform dynamic time warping to at least one aggregate sensor data of the array of aggregate sensor data; and
compare each dynamic time warped data to the recording file; and
generate an indicator based on the comparison.

5. The system of claim 1, further comprising: a peripheral device in communication with the base pod, the peripheral device configured to receive an aggregate of the sensor data, compare the aggregate of the sensor data to an Xpression library having one or more Xpressions, and determine when a match is made between the aggregate of the sensor data and the one or more Xpressions.

6. The system of claim 1, wherein the base pod is wirelessly connected to the one or more distributed modules.

7. The system of claim 1, wherein the base pod is physically connected to the one or more distributed modules.

8. The system of claim 1, wherein at least one of the one or more sensors is configured to provide a biological attribute of the wearer.

9. The system of claim 1, the system further comprising a peripheral device in communication with the base pod, the peripheral device configured to receive an aggregate of the sensor data, compare the aggregate of the sensor data to an Xpression library having one or more Xpressions, and to determine when a match is made between the aggregate of the sensor data and the one or more Xpressions.

10. A method for generating a recording file defining a tacit motion, the method comprising:
receiving, by one or more distributed modules, sensor data from one or more sensors distributed at distinct locations relative to a moving body;
generating, by each distributed module, an aggregate sensor data from the sensor data;
communicating, by each distributed module, the aggregate sensor data to a base pod;
receiving, by the base pod, an array of aggregate sensor data from the one or more distributed modules;
generating a header defining an attribute of each distributed module and allowing for compiling of the array of aggregate sensor data; and
generating a recording file having the header and the array of aggregate sensor data.

11. A method for generating an Xpression associated with a tacit motion, the method comprising:
receiving an array of aggregate sensor data from at least one base pod and at least one distributed module, and at least one of attribute of each base pod and each distributed module;
identifying, for each aggregate sensor data contemporaneously within the array of aggregate sensor data, one or more patterns of coordinated sensor data across each base pod and the one or more distributed modules;
generating a framework using at least one of the attributes of each base pod and each distributed module and the one or more patterns of coordinated sensor data; and
determining at least one metric describing the array of aggregate sensor data based on the framework.
